(19) United States
(12) Patent Application Publication
(10) Pub. No.: US 2025/0152903 A1
(43) Pub. Date: May 15, 2025

(54) CHEMICAL-BASED NITRIC OXIDE GAS-GENERATING DRUG DEVICE FOR DELIVERY TO A PATIENT

(71) Applicant: THE UAB RESEARCH FOUNDATION, BIRMINGHAM, AL (US)

(72) Inventors: Nagababu ENIKA, Birmingham, AL (US); Dan E. BERKOWITZ, Mountain Brook, AL (US); Lakshmi SARAVANKUMAR, Birmingham, AL (US)

(21) Appl. No.: 18/838,187

(22) PCT Filed: Feb. 14, 2023
(86) PCT No.: PCT/US2023/062609

§ 371 Date: Aug. 13, 2024

Related U.S. Application Data
(60) Provisional application No. 63/309,889, filed on Feb. 14, 2022.

(51) Int. Cl. A61M 16/12; A61M 16/00; A61M 16/10; A61M 16/20

(52) U.S. Cl. CPC A61M 16/122; A61M 16/0051; A61M 16/1005; A61M 16/203; A61M 2016/0027; A61M 2016/0039; A61M 2202/0275; A61M 2230/43

(57) ABSTRACT

Provided are methods for delivering NO to patients, systems for delivering NO to patients, devices for generation and storage of NO, and methods for generating NO. Described are systems providing single-step and dual-step dilution of high concentration NO for delivery to a patient without generating toxic levels of NO₂. Also described are NO storage cartridges for use in the systems.

### CROSS-REFERENCE TO RELATED APPLICATIONS

This application claims the benefit of and priority to U.S. Provisional Application Ser. No. 63/309,889, having the title “CHEMICAL-BASED NITRIC OXIDE GAS-GENERATING DRUG DEVICE FOR DELIVERY TO A PATIENT”, filed on Feb. 14, 2022, the disclosure of which is incorporated herein by reference in its entirety.

### BACKGROUND

Inhaled nitric oxide (iNO) is used as a pulmonary-specific vasodilator without compromising the systemic blood pressure. Inhaled nitric oxide is used to treat persistent pulmonary hypertension associated with hypoxic lung failure in infants and life-threatening pulmonary hypertension in children and adults. Current tank-based iNO therapy is complex, expensive, and generally only available in the intensive care units and operating rooms of established medical centers. To make this therapy available to a broader patient population, less complex, less expensive, and non-tank portable iNO therapy devices are needed that can be used in and out of hospitals whenever and wherever they are needed.

### SUMMARY

In accordance with the purpose(s) of the present disclosure, as embodied and broadly described herein, embodiments of the present disclosure, in some aspects, relate to methods for delivering NO to patients, systems for delivering NO to patients, devices for generation and storage of NO, and methods for generating NO.

The present disclosure includes a device for on-demand generation of NO which can include a reaction vessel and a liquid storage component, wherein the reaction vessel comprises a liquid injection port and a gas exit port. The liquid injection port and a gas exit port are each fitted with a self-sealing plug connector that is pressure-tolerant up to 500 PSI, such that the reaction vessel is self-sealing and pressure-tolerant up to 500 PSI. The liquid storage component is removably coupled to the reaction vessel at the liquid injection port via a socket connector.

Embodiments of the present disclosure include a two-stage dilution system for delivering NO to a patient. The system can include a removable NO gas cartridge in fluid communication with a gas mixing chamber and a CPU. The gas mixing chamber can include a carrier gas inlet, an NO inlet, a mixing space, and a gas outlet, wherein the NO inlet comprises an NO nozzle having an internal diameter of about 10 μm to 50 μm. The concentrated NO gas is delivered from the NO gas cartridge to the gas mixing chamber via a gas manifold, and the pressure of the NO gas in the manifold is controlled by an electronic pressure regulator in communication with the CPU. A carrier gas selected from air or N₂ flows into the carrier gas inlet at a fixed flow rate. The NO nozzle injects the NO into a stream of carrier gas to mix the gas in the mixing chamber to form a first diluted mixed gas comprised of NO diluted in the carrier gas. The mixed gas comprises less than 1000 ppm of NO and less than 25 ppb of NO₂. In a second dilution step, an oxygen source is coupled to the gas outlet via a flow sensor. The flow sensor measures the flow rate of inspired O₂ and communicates to the CPU to inject the desired NO dose of the first diluted mixed gas into the O₂ for delivery of a second dilute gas to a patient via an inspiratory tube at a desired NO dose, and wherein the desired NO dose is about 1 ppm to 80 ppm.

Embodiments of the present disclosure also include a single-dilution system for delivering NO to a patient, the system as above, but wherein the NO nozzle injects the NO into a stream of carrier gas to mix the gas in the mixing chamber to form a mixed gas comprised of NO diluted in carrier gas, wherein the mixed gas comprises from 1 ppm to 1000 ppm, and wherein the mixed gas is delivered to the patient without the second dilution.

Other compositions, apparatus, methods, features, and advantages will be or become apparent to one with skill in the art upon examination of the following drawings and detailed description. It is intended that all such additional compositions, apparatus, methods, features and advantages be included within this description, be within the scope of the present disclosure, and be protected by the accompanying claims.

### BRIEF DESCRIPTION OF THE DRAWINGS

Further aspects of the present disclosure will be more readily appreciated upon review of the detailed description of its various embodiments, described below, when taken in conjunction with the accompanying drawings.

FIGS. 1A and 1B are schematic views of a cartridge for storing chemicals and liquids used in NO generation in accordance with embodiments of the present disclosure.

FIGS. 2A and 2B are graphs demonstrating rates of NO generation in the cartridge where the chemical mixture of N-acetylcysteine (NAC), sodium nitrite, and cuprous chloride [Cu(I)Cl] is reacted with water under nitrogen (N₂) and oxygen (O₂) gases at 1 ATM pressure and 0.3 ATM created by vacuum.

FIG. 3 is a graph comparing the NO₂ levels in 100 ppm NO generated under air and N₂ conditions without stirring the sample, demonstrating sample purity.

FIG. 4 is a graph demonstrating that the unstable NO generated in the reaction vessel is stabilized by raising the pH from acidic to neutral.

FIG. 5 is a graph demonstrating that the inclusion of an additional Na₂HPO₄ compound into the NO-generating chemical mixture to raise the pH partially decreases the amount of NO generated.

FIG. 6 is a graph demonstrating that inclusion of Na₂HPO₄ in the NO-generating mixture slows the rate of NO generation.

FIG. 7 is a graph demonstrating the stability of chemicals under N₂ and O₂ at 1 ATM pressure in gastight sealed tubes.

FIG. 8 is a graph showing the conversion of nitrite to NO by ascorbic acid and Cu(II)Cl₂ in aqueous solution.

FIGS. 9A-9D are diagrams illustrating the mixing chamber and its assembly used to control NO delivery and for rapid mixing of NO with carrier gas (O₂) to minimize the NO₂ formation in the iNO device in accordance with embodiments of the present disclosure. FIG. 9A shows an embodiment of the mixing chamber. FIG. 9B is a close-up of the NO inlet shown in FIG. 9A. FIG. 9C is a close-up of the carrier gas inlet. FIG. 9D is a close-up of the mixing chamber at the mixing point of NO and carrier gas. FIG. 9E is diagram of a prototype mixing chamber.

FIG. 10 is a graph demonstrating the NO₂ formation during the mixing of NO with O₂ using a nozzle-mixing chamber in accordance with embodiments of the present disclosure.

FIG. 11 is a camera image of a benchtop iNO delivery system that simulates a patient NO delivery system and is used to determine the NO and NO₂ levels in the therapeutic inhaled NO gas before and after (trachea) inhalation in accordance with embodiments of the present disclosure.

FIG. 12A is a graph showing the correlation of the gas pressure difference between the manifold (NO gas-line) and mixing chamber (carrier gas) with NO dose in accordance with embodiments of the present disclosure. FIG. 12A plots the NO concentration against the pressure difference. FIG. 12B plots the carrier gas flow rate against the pressure difference.

FIG. 13 is a diagram illustrating the process of dosing of NO based on the pressure difference between NO gas and inspired oxygen in accordance with embodiments of the present disclosure.

FIG. 14 is a schematic diagram of an embodiment of an iNO device based on the NO dosing with the pressure difference of NO and inspired oxygen to use in hospitals.

FIGS. 15A and 15B are graphs demonstrating that the pressure difference of the system is proportional to NO dosing, in accordance with embodiments of the present disclosure.

FIGS. 16A and 15B are graphs showing dose modulation during iNO therapy in accordance with embodiments of the present disclosure.

FIG. 17 is a graph showing that the pressure difference is correlated to carrier gas flow rate at the fixed NO dose.

FIG. 18 is a schematic diagram showing dosing of NO using a proportional valve system in accordance with embodiments of the present disclosure.

FIG. 19 is a diagram of an example of the architecture of a proportional valve in the mixing chamber.

FIGS. 20A and 20B are graphs demonstrating that the proportional valve pulse width time is proportional to NO dose.

FIGS. 21A and 21B are graphs demonstrating NO dose change by modulation of valve pulse width time during therapy.

FIG. 22 is a graph showing that the carrier gas flow rate is proportional to valve pulse width rate.

FIG. 23 is a diagram of an example of a portable iNO delivery system.

The drawings illustrate only example embodiments and are therefore not to be considered limiting of the scope described herein, as other equally effective embodiments are within the scope and spirit of this disclosure. The elements and features shown in the drawings are not necessarily drawn to scale, emphasis instead being placed upon clearly illustrating the principles of the embodiments. Additionally, certain dimensions may be exaggerated to help visually convey certain principles. In the drawings, similar reference numerals between figures designate like or corresponding, but not necessarily the same, elements.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of anesthesiology, molecular medicine, chemistry, material science, and the like, which are within the skill of the art.

The following examples are put forth so as to provide those of ordinary skill in the art with a complete disclosure and description of how to perform the methods and use the systems disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

As used herein, the following terms have the meanings ascribed to them unless specified otherwise. In this disclosure, “consisting essentially of” or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein. “Consisting essentially of” or “consists essentially” or the like, when applied to methods and compositions encompassed by the present disclosure have the meaning ascribed in U.S. Patent law and the term is open-ended, allowing for the presence of more than that which is recited so long as basic or novel characteristics of that which is recited is not changed by the presence of more than that which is recited, but excludes prior art embodiments.

### Definitions

Proportional valve, as used herein, refers to programmable electronically-controlled valves in which the flow speed and directionality of fluid are controlled through electronic pulsing of the valve opening. These valves do not require a pressure drop to operate.

Carrier gas, as used herein, can refer to nitrogen, air, air blended with oxygen (e.g., 21 to 80), or oxygen (21 to 100%) administered to a patient according to need.

### Abbreviations

Nitric oxide (NO); inhaled nitric oxide (iNO); Nitrogen dioxide (NO₂); N-acetylcysteine (NAC); S-nitroso-N-acetylcysteine (SNOAC); cuprous chloride (Cu(I)Cl; Cupric chloride (Cu(II)Cl₂: Disodium biphosphate (Na₂HPO₄)

### General Discussion

Pulmonary hypertension (PH) is a highly debilitating disease, and no specific drugs are available for pharmacologic treatment. Inhaled NO (iNO) is a gold standard to provide a pulmonary specific vasodilator without compromising systemic blood pressure for treating/managing acute PH. Several clinical studies show that iNO clearly improves the ventilation-perfusion matching and lowers the pulmonary vascular resistance, thereby improving the oxygenation of blood in pulmonary arterial hypertension (PAH) and several PH-associated lung diseases. Presently, iNO has been used for the treatment of persistent pulmonary hypertension/hypoxic lung failure in infants and managing hypoxic lung failure and life-threatening pulmonary hypertension for adults to avoid the need for more invasive and expensive Extracorporeal Membrane Oxygenation (ECMO) treatment.

Current iNO therapy requires a complex and expensive (approximately $180/hour) tank-based NO delivery systems. Therefore, this therapy is available only in the intensive care units and operating rooms of established hospitals. It is possible that impurities in NO and the use of statistically underpowered subjects in clinical trials may be responsible for not observing clinical benefits of chronic treatment. Hence, there is a need for developing iNO therapy devices that are portable, less complex, less expensive and a desired amount of on-demand medical grade NO generation systems. The present disclosure addresses this need and other needs.

Since the FDA approved NO gas as a pharmaceutical agent, efforts are being made to develop portable NO-generating systems. Nitrite can be converted to NO in aqueous solutions, using mildly acidic conditions, electrolysis, high temperature, UV irradiation, electrochemical reduction using of Cu(II) to Cu(I) and ascorbic acid as a reducing agent, The major problem in these procedures is removing NO gas from the reaction vessel without generating toxic gases before introducing it to carrier gas. Catalytic reduction of highly toxic liquid N₂O₄/NO₂ to NO is also being developed as a source of iNO. Other investigators are actively pursuing converting atmospheric air (N₂ and O₂) to iNO using high electric discharge electrodes. This procedure also generates toxic gases like NO₂, ozone, other reactive oxygen species, and particulate matter in addition to NO. These need to be removed completely before administering to patients. Other start-up companies like Bellerophon pulse Technologies, Warren, NJ, Nu-Med Plus, Inc. Salt Lake city. UT and Novoteris, Canada have been developing portable NO delivery devices using a concentrated NO (2500 to 5000 ppm) blended with N₂ in mini-cylinders, which can last only for a few hours. In these systems NO₂ is also expected to be a major contaminant during storage and diluting the stock NO to therapeutic doses. In addition, patients need to buy NO cylinders from pharmacies as they do prescription medicine. All of these obstacles add to the cost of treatment. As such, there is a need for development of better methods for generating NO as well as better systems for delivering NO.

Therefore, the technology of the present disclosure for NO generation is focused on simple methods for generation of on-demand NO that are inexpensive and without need for purification. The treatment can be available to a wider global population for use both in and out of hospital facilities. The systems and methods described herein can be used for such as ambulatory patients, in a patient's home, and in hospitals or clinics, including while patients are transporting within or between facilities.

The methods and systems described herein present improvements in iNO therapy. The systems provided simplify the both the process for diluting NO with supplemental oxygen to the therapeutic doses and the process for delivery. Amongst other improvements, the present system introduces an electronically controllable pressure regulator for dosing NO based on the pressure difference between NO and NO carrier gas.

Two types of iNO systems are described herein. In a first system, intended mainly as an alternative to existing systems used in clinical settings. This system uses a dual dilution process in which high concentration NO is first diluted in a carrier gas through a controlled nozzle and flow configuration that prevents the formation of toxic levels of NO₂ gas. The diluted gas, also referred to as mixed gas, is then diluted a second time as it is mixed with inspired oxygen at the point of delivery to a patient. Advantageously, the system allows for diluted, accurate dosing of NO from highly concentrated NO stock on demand. The NO can be diluted by a factor of about 40,000 while minimizing the formation of NO₂. The NO₂ produced using the system is less than 100 ppb, which is approximately 10 times less than the maximum allowed amount by the FDA, resulting in a very safe delivery system.

In this system, toxic NO formation is reduced at two stages where NO₂ formation can potentially formed. In any reaction, the mixing point of NO with O₂ is the source of about 99% of NO₂ formation, especially when mixing highly concentrated NO. However, the present system uses a controlled nozzle system in a mixing chamber to minimize the formation of NO₂ by rapid dilution The NO is diluted in the mixing chamber into a carrier gas comprising either N₂ or O₂ (21 to 100%) O₂ gas. The carrier gas is pumped into the mixing chamber at a fixed flow rate of carrier gas (N₂ or air). A gas flow rate sensor in the inspiratory tube controls the desired NO dose from the exit of the mixing chamber into the oxygen flowing to the patient's inspiratory tube. The diluted NO (<1000 ppm) is injected into inspired into inspiratory tube through injector nozzle having an orifice from about 50 microns to about 500 microns. In some embodiments, NO is directly injected into the inspired oxygen in the mixing chamber. The gas exit from the mixing chamber is directly used for inhalation. In various embodiments, the system can also include a gas sampling line in communication with one or more gas analyzers to measure NO and//or NO₂ to ensure patient safety.

In a second system, intended mainly to be used as a portable system for use outside of clinical settings (e.g., home use, during patient transportation, or in emergency situations), the high concentration NO or diluted NO is diluted in a single or two or three doses into inspired oxygen immediately prior to delivery to the patient. In this embodiment, the carrier gas used in the mixing chamber is mainly atmospheric air or O₂ gas. The NO flow to the mixing chamber is controlled by electronically regulated pressure regulator or proportional valves placed between the NO cartridge and the mixing chamber.

In both systems, the dose administered to the patient remains constant through control of the pressure of NO gas entering the carrier gas stream through the NO nozzle. The dose can be set through a user interface connected to a CPU, and the CPU controls the pressure of NO exiting the nozzle via a series of sensors and pressure regulators.

In various embodiments, the NO is injected into the carrier gas stream inside a mixing chamber via an NO delivery nozzle having a micro-orifice. The carrier gas (air or N₂ in the dual-dilution system, O₂ in the single dilution system) enters the mixing chamber through a carrier gas inlet, forming a stream of carrier gas. NO is injected into the stream. The nozzle system is used as a means to minimize the nitrogen dioxide (NO₂) formation as well as control the injection of NO in microliter volumes into NO carrier gas. These improvements and others disclosed herein make the device simpler, smaller, lighter weight and better able to provide precise dosing for use in and out of hospitals. In some embodiments, concentrated NO gas (such as from about 2% to about 100% concentration) can be delivered into the carrier gas stream by a nozzle having an exit diameter of about 2.5 μm to 100 μm (e.g., 2.5 μm, 5 μm, 10 μm, 25 μm, 50 μm). Advantageously, the small nozzle size creates a build up of NO pressure in the manifold so that the NO can be metered. The small nozzle size also mixes NO rapidly with O₂ rapidly so that NO₂ formation can be minimized.

In some embodiments, the mixed gas (e.g. the NO diluted in the carrier gas) has a concentration of about less than 0.1% and is delivered from the device by an outlet having an exit diameter of about 50 μm to 1000 μm (e.g., 50 μm, 100 μm, 150 μm, 200 μm, 250 μm, 500 μm, 750 μm, 1000 μm).

In some embodiments, the carrier gas can be delivered to the mixing chamber at a flow rate of about 2 L/min to 12 L/min, or about 5 L/min to 8 L/min.

In various embodiments, the NO dosing is controlled using the pressure difference between the carrier gas and the NO gas. In this manner, the dosing remains constant even where the carrier gas flow or pressure changes. Where the carrier flow increases, the pressure difference also increases. Given a specific NO dose for desired delivery to a patient, a known NO concentration provided the system, and a known carrier gas flow rate, a pressure difference is established. Should the carrier gas pressure or flow rate change, the pressure of the NO dose delivered to the carrier gas can be proportionately adjusted to maintain the NO dose. In an illustrative non-limiting example, where a health care provider prescribes a NO dose of 25 ppm, with a typical 8 L/min carrier gas flow, there is a pressure difference of 12 kPa between the NO pressure and the carrier gas pressure. If the carrier gas flow was then reduced to 5 L/min, a pressure difference of 5 kPa would be needed to maintain the 25 ppm dose rate. The system would adjust to reduce the pressure of NO to achieve the 5 kPA pressure difference, thereby delivering the 25 ppm dose to the patient despite the change in gas flow rate. So, in this way the system can compensate if the flow rate changes to keep the dose constant. Similarly, if the user adjusts the desired dosage amount, the system can adjust the proportion of NO needed to achieve the dosage based on the carrier gas flow rate.

Where NO dosing is controlled using the pressure difference method described above, the NO is delivered from the NO source (such as a cartridge as described below) via a gas manifold, through the NO nozzle into the mixing chamber. The pressure and flow rate are controlled by an electronic NO pressure regulator and a carrier gas flow rate and pressure sensor in communication with a CPU. In some embodiments, the carrier gas flow is further controlled by a solenoid valve.

In another embodiment, the NO dosing is controlled by an electronically controllable proportional valve. Here, an ultra-miniature proportional valve is used in conjunction with the small (e.g., 10 or 25 micron) delivery nozzles to deliver the NO to the mixing chamber. Instead of the dosing being based on pressure difference as above, the pressure of the NO in the gas manifold remains constant and higher than the carrier gas pressure. The manifold pressure is set at lowest possible value that maintains a positive differential pressure between manifold and mixing chamber (across all anticipated carrier gas pressures) and is maintained by a pressure regulator. The pressure can be maintained at a limit such as less than 30 psi. The dosing is controlled by the amount of NO emitted through proportional valve. The proportional valve pulse width determines the amount of NO, where a high pulse width delivers less NO and a low pulse width means that the valve is open longer and therefore delivers more NO. The valve pulse width time is proportional to the NO dose. The valve pulse width time is proportional to the carrier gas flow rate at the fixed, prescribed NO dose.

In various embodiments, a system equipped with a proportional valve can be operated based the pressure differential method as described above. When the proportional valve is left open, the system can function based on the pressure differential. In this way, the pressure in the manifold is no longer constant, but is instead managed by the pressure regulator.

In various embodiments, the mixing chamber includes a carrier gas inlet and an NO inlet entering a mixing space, and a gas exit port exiting the mixing space. In some embodiments, the mixing space can have a volume of about 10 mL to about 50 mL, or about 20 mL. The NO inlet extends into the mixing chamber via a nozzle assembly that includes a hypodermic stainless-steel tube. In some embodiments the tube can be a 13-gauge tube. The tube is fitted with a nozzle having an internal diameter of 2.5 μm to 50 μm. The NO nozzle can have, in some embodiments, a corundum orifice. In a particular embodiment, the NO nozzle has a 10 μm internal diameter opening. The NO inlet is configured such that the NO injected through the nozzle enters into a stream of carrier gas entering the mixing space through the carrier gas inlet. The now combined stream of NO and carrier gas then encounters an obstruction which diverts the combined gas around the mixing space to mix it further. The carrier gas inlet and the gas exit port have the same diameter, causing the mixing chamber to operate under steady-state pressure such that the pressure of the gases entering is the same as the pressure of the combined gas exiting through the exit port.

In general, the NO gas is provided to the systems described by a NO cartridge in which NO is both generated and stored. This cartridge is then inserted into the system such that the NO from the cartridge is mixed in a mixing chamber with carrier gas for delivery to a patient. The concentration and flow velocity of NO and flow velocity of carrier gas is precisely controlled while mixing them together such that the formation of toxic NO₂ is minimized while providing the patient with a prescribed NO dosage.

The NO is delivered to the system from an NO cartridge. The cartridge includes two primary components. The first component is a reaction vessel, which is used for storage of NO-generating dry chemicals prior to reaction and for storage of the high concentrated NO gas generated by reacting the chemicals with water. It can be a pressure-resistant, nonreactive material such as stainless steel. The reaction vessel can have a capacity of about 50 mL to about 200 mL. The reaction vessel has two ports, one for injecting liquid and/or inserting the reagents, and one for gas exit when the reaction vessel is inserted into one of the systems above. Each of the ports can be fitted with self-sealing plug connectors that are pressure tolerant (e.g., to about 500 psi) such that the reaction vessel is self-sealing and pressure resistant.

The second component is a liquid storage component. The liquid storage component delivers a liquid reactant such as water to the reaction vessel through the liquid injection port and is then removed. Upon injection of the liquid, the NO reaction commences to form NO gas. The pressure from the gas pressurizes the reaction vessel. In some embodiments, the liquid storage component can be a pressure-resistant, gas-tolerant syringe for manual injection of the fluid. In other embodiments, the liquid storage component can be a pressure-resistant, gas-tolerant vessel where the liquid is added to the reaction vessel when the NO-generating chemicals are stored under vacuum. The liquid storage component can be removably coupled to the reaction vessel's injection port by a socket coupler (e.g., a luer lock or other suitable connector) that provides a gas-tight connection between the two components. The socket can couple with a plug connector on the reaction vessel injection port. Advantageously, the reaction vessel can be recycled or reused. The liquid storage component can be disposable or reusable. Advantageously, the liquid storage component can be pre-filled with an amount of liquid precisely measured for the reactants in the reactant vessel.

The NO gas in the cartridge can have a concentration of about 2% to about 100%. In some embodiments, the NO gas can be diluted to about 2% to 50% by N₂ gas in the cartridge for use in such as portable systems, providing NO supply appropriate for short-term (e.g., 2 to 24 hours.) usage. In other embodiments, the NO gas can be stored undiluted in the cartridge at a high concentration such as 80%, 90%, or higher for use in the system.

Also provided herein are various methods for generating high-purity, high concentrations of NO gas. In a particular embodiment, reactants sodium nitrite, N-acetylcysteine powder and cuprous chloride crystals in a ratio of 1:1:0.2 are stored in the reaction vessel. Advantageously, the completely dry reactants can be stored in the completely moisture and O₂ free conditions of the cartridge described above for up to two years without affecting stability. The NO-generating reaction is initiated by addition of water from the liquid storage component. The reaction occurs under anaerobic conditions. Advantageously, because the reactants in the vessel are in powder form, the reaction occurs without mixing or agitation. One hundred percent of the nitrite is converted to NO. About 80% of this generation occurs within one minute. The reaction can be generated under N₂ pumped into the reaction vessel through the exit port to reduce potential reaction with O₂ in the vessel. The presence of any trace oxygen reacts with NO to forms NO₂, but this NO₂ diffuses back into water and converts to nitrous acid and nitrite, resulting in the final NO generated in the reaction vessel being 99.7% pure. Advantageously, this fast, pure reaction can be initiated on-demand, such as where NO intervention is needed in emergency situations.

In some embodiments, the reactants include sodium nitrite, NAC, Cu(I) chloride and Na₂HPO₄ in a ratio of 1:1.25:0.2:1.25. The addition of Na₂HPO₄ increase the shelf-life and stability of the generated NO, although the yield is slightly lowered.

In other embodiments, the reactants can be sodium nitrate, NAC, and Cu(II) chloride, and a proton donor. In some embodiments, the proton donor is ascorbic acid. In other embodiments, the proton donor can be a carboxylic group of NAC, substrate itself. Other proton donors can include, but are not limited to, ascorbyl palmitate, salicylic acid, malic acid, lactic acid, citric acid, formic acid, benzoic acid, tartaric acid, hydrochloric acid, sulfuric acid, and phosphoric acid.

### Examples

Now having described the embodiments of the disclosure, in general, the examples describe some additional embodiments. While embodiments of the present disclosure are described in connection with the example and the corresponding text and figures, there is no intent to limit embodiments of the disclosure to these descriptions. On the contrary, the intent is to cover all alternatives, modifications, and equivalents included within the spirit and scope of embodiments of the present disclosure.

### Systems for NO Generation and Delivery

Devices and methods for portable delivery of iNO are discussed in PCT/US2021/071242, herein incorporated entirely by reference. The present invention can be used for storing chemicals and generating NO gas on-demand in the NO-cartridge. The present invention provides improved chemical storage stability as well as NO gas stability after generation, more controlled metering (dosage) of NO into a carrier gas, and higher NO purity. The present disclosure provides for systems and methods for bulk concentrated (e.g., 90 to 98%) NO generation and delivery. In general, an NO-generating chemical reaction is performed in a cartridge. The cartridge can be immediately placed in the delivery system for delivery to a patient or can be stored for later use for an extended period (such as 3 months) in the delivery system. The following examples describe the cartridge and the chemistry for generating the NO as well as the system for delivering the generated NO. The chemistry, cartridge, and system work in tandem to deliver iNO therapy to the patient.

Here, the cartridge, has been modified and simplified. First, a known amount of 90 to 98% of NO is generated in a cartridge. The NO is also diluted with nitrogen to a desired percent level using high pressure nitrogen tanks. The assembly of the cartridge's reaction vessel with high pressure threading self-sealing NPT connectors makes it safe for generating on-demand NO. Then, the cartridge is inserted into a receptacle in the iNO system for passage of NO into a mixing chamber. Advantageously, the cartridge allows simple, safe generation of NO by any healthcare worker, patient, caregiver, or other non-technical individual. The cartridge is also recyclable. This cartridge improves chemical compatibility and pressure thresholds associated with the previously disclosed device. The cartridge described herein generates on-demand, pressurized NO with a purity of about 90% to 99.6% within the cartridge by a chemical method.

In embodiments, prior to NO generation, the atmospheric air in the cartridge is replaced with nitrogen and evacuated. The evacuation does not remove all of the nitrogen. About 0.3% (30 ml in 100 ml reaction vessel) nitrogen is retained in the cartridge. This nitrogen dilutes the generated NO gas, resulting in a percentage of NO about 90% to 98%. The percentage of dilution is dependent on how much NO is generated. Depending on need and the amount of reagents, about 0.27 L (50 psi) ml to 1.5 L (230 psi) may be generated. The self-sealing connectors allow for safe storage of concentrated NO at high pressure for subsequent dosing. Other non-tank delivery systems generate very low concentration NO (about 0.08% (800 ppm) on site. Any changes in the desired dose requires a change in the chemical synthesis of the NO (Vero-biotech, LLC, Atlanta, GA, Beyond Air, Inc. New York, and Nota labs. Inc. Ann Arbor, MI). In contrast, the present system allows for high concentration (10% to 100%) pressurized NO in which the dosage can be altered in seconds based on metering the NO with carrier gas flow rates.

In the previous disclosure, it was proposed that the NO-generating chemicals be formulated as a tablet or capsule to increase the stability and shelf-life and to enable storage in an aerated (21% O₂) NO cartridge. A magnetic stirrer that was integrated within the device was used to promote dissolution of the solid chemicals into the liquid. The stirring also facilitated diffusion of NO₂ from the reaction vessel headspace to the solution to convert back to NO. In the present modified device, we have omitted the magnetic stirrer and programmatically actuated mechanism for combining the chemicals with liquid and transferring NO to the reservoir. Instead, NO is generated in a cartridge (described below) before inserting the cartridge into a receptacle of the iNO for passage of NO into a reservoir or mixing chamber. In the present system, chemicals in fine powder form are used because they dissolve or suspend quickly in the aqueous solution to minimize the need for stirring. The moisture content is also reduced in the gas. Further details are provided in the examples below.

### Cartridge for NO Storage and Generation

The cartridge 100 is a self-sealing, pressure resistant vessel for storage of NO-generating chemicals. Cartridge 100 includes a reaction vessel 110 that is configured to be self-sealing. Reaction vessel 110 can store either unreacted first reactants or fully reacted NO. Reaction vessel 110 is configured to removably couple with liquid storage component 120. Once water is injected from liquid storage component 120 to initiate a reaction in reaction vessel 110, liquid storage component 120 can be released from reaction vessel 110. Reaction vessel 110 self-seals such that the reaction can take place inside, creating a pressurized vessel. Advantageously, the cartridge is very safe; it can be dropped, handled, moved, etc. without releasing the contents or exposing the contents to air.

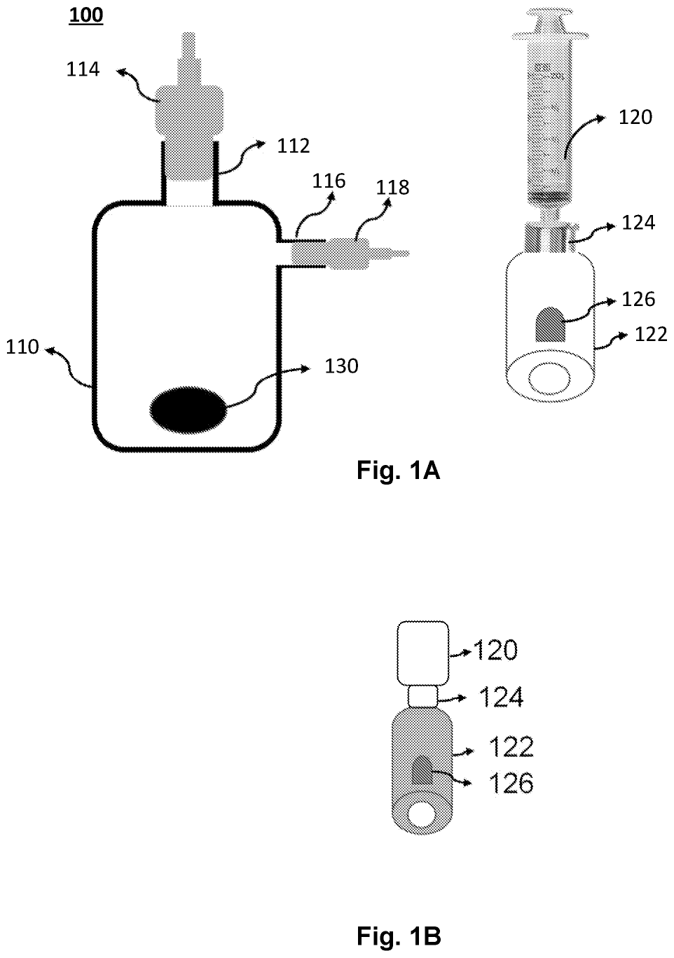

Example cartridges are illustrated in FIGS. 1A and 1B. The vessel 110 liquid storage component 120, shown as a pressure resistant gas-tight syringe in FIG. 1A, stores the liquid portion of the reaction mixture. The syringe plunger is depressed to inject the liquid into the reaction vessel 110. The reaction vessel 110 is compatible with the chemical reagents and high gas pressure. Quick-connect and quick-disconnect self-sealing plug and socket couplers (Staubli, Duncan, SC) are used to combine reactants to trigger the NO-generating reaction wherever and whenever it is needed. Non-spill connectors and disconnectors can also be used as needed. The reaction vessel 110 shown in FIG. 1A has a 50 to 100 ml capacity. Liquid injection port 112 is a port for inserting NO-generating reagents 130 and injecting liquid from liquid storage component 120. In an embodiment, the liquid injection port 112 is a ¼-inch female national pipe thread (FNPT). After reagents 130 are added to the vessel, liquid injection port 112 is closed with a self-sealing plug connector 114. In an embodiment, the plug connector 114 is a ¼-inch male national pipe thread (MNPT) plug connecter that tolerates gas pressure up to 500 psi. Reaction vessel 110 includes a gas exit port 116 in fluid communication with a gas exit port 116 equipped with a plug connector 118. In an embodiment, gas exit port 116 is a 1/16 or ⅛-inch FNPT port closed with a 1/16- or ⅛-inch MNPT plug connecter 118.

Liquid storage component 120 is removably coupled to reaction vessel 110 by a socket coupler 122 that pairs with plug connector 114 when both components are fitted together. Socket coupler 122 is provided an airtight connection to the reactant vessel 110 by coupler 124. Socket coupler 122 can be released from reaction vessel 110 by release button 126. In an embodiment, coupler 124 is a luer lock that forms an airtight connection to the storage chamber 120, forming a custom modification for socket coupler 122.

FIG. 1B provides an alternative embodiment of liquid storage component of reactant vessel 120. In this embodiment, the liquid is provided to the reaction vessel 110 by suction applied to gas exit port 116. The reactant vessel 120 is a pressure resistant (500 psi) custom made stain-less steel vessel with ⅛ FNPT inlet. The vessel is connected to the original socket's ⅛-inch MNPT connector. These connectors are tolerable to the pressure up to 500 psi. The reaction vessel has a safety feature if the socket releasing button fails. The NO retains in the vessel without releasing out. The connectors between the reaction vessel 110 and socket 122 are tolerant to the pressure of 500 psi.

Liquid storage component 120 can be prefilled with an appropriate amount of a liquid diluent. When NO is needed, the socket coupler 122 is snapped to the plug connector 114, creating a quick connection to make a passage between the reaction vessel 110 and liquid storage component 120. The socket coupler 122 can be disconnected quickly by pressing the release button 126. After injecting liquids, the gas is generated and pressurizes the reaction vessel. This pressure can push the syringe plunger of liquid storage component 120 to release NO out if the socket coupler 122 is not removed. In some embodiments, a plunger flange is made to lock the socket 122, so that the pressure does not push the plunger out to release gas.

Alternatively, the process of mixing liquid with the chemicals in the cartridge 100 can be automated after insertion of the cartridge 100 into an assigned receptacle of the larger iNO system, where the liquid storage component 120 is part of the iNO system. A small amount of liquid can remain in the plug connector 114 during the transfer of liquid into reaction vessel 110. Hence, liquid injection port 112 is not used as a dual-purpose gas exit port and liquid injection port to avoid liquid contamination in the gas. The moisture content in the NO collected from the gas exit port 116 is less than 0.2% because the stirring of reagents is not required to initiate the reaction. This cartridge 100 has the ability to make up to 1.5 L of 100% pure NO gas having a pressure of about 50 psi to about 230 psi based on the requirement. In addition, the NO generated in the reaction vessel 110 can be diluted (e.g., about 10% to 90%) with nitrogen gas to use in inhaled NO therapy.

### NO Generation Methods

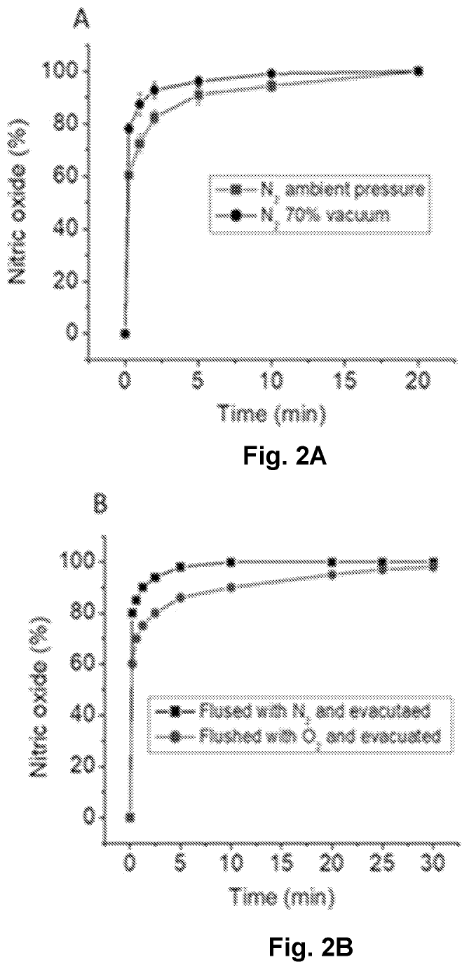

Turning now to FIGS. 2A and 2B, the rates of NO generation in the cartridge under nitrogen (N₂) and oxygen (O₂) gases at 1 ATM pressure and 0.3 ATM created by vacuum are shown. The reason for investigating NO generation under N₂ and O₂ was to ascertain the effect of vacuum and gases on the rate of NO generation and purity of NO and apply these procedures in cartridge development. After placing the chemicals in the reaction vessel 110, the vessel is closed. At this point, the reaction vessel 110 contains atmospheric air. This air is flushed with nitrogen gas to replace the air with nitrogen. Then the nitrogen gas is removed by a vacuum pump by connecting plug 114 with a vacuum pump. The vacuum pump can remove about 70% of nitrogen. If the experiment results show that air in the reaction vessel 110 does not interfere with NO generation or increase NO₂ formation, this step could be omitted in the system.

The NO-generating chemical mixture (discussed in detail below) was placed in the reaction vessel and then subjected to N₂ at standard atmospheric pressure (1 ATM), N₂ at 70% vacuum (0.3 ATM) (FIG. 2A), and O₂ at 70% vacuum (FIG. 2B). The reaction was initiated by introducing the liquid and the rate of NO generation was measured with time and expressed as a percentage of the expected value. The NO₂ levels in this NO gas were also measured by Cavity Attenuated Phase Shift (CAPS) NO₂ analyzer. In all these conditions, exactly 500 ml of NO was produced albeit at different rates. All nitrite was converted to NO on moles basis at the nitrite, NAC and Cu(I) ratio of 1:1:0.2, respectively. We were not able to measure the differences in the initial rates under varying conditions because of the rapid release of NO. As shown in FIGS. 2A and 2B, approximately 90% of nitrite was converted to NO within 5 minutes in all of the experimental conditions without stirring or mixing of the reagents. Advantageously, reaction mixture stirring was not required to initiate the reaction or generate NO, illustrating that NO can be generated outside of the iNO system using the cartridge described above. Thus, there is no need to integrate the device with a magnetic stirrer or to use a computer to programmatically actuate the mechanism to mix and transfer NO to the reservoir in the iNO system. The rate of NO generation is faster in the N₂-evacuated sample than in the air-evacuated sample. To understand the effect of the vacuum on the reaction rate, we compared the rate of NO generation under N₂ at standard atmospheric pressure to that under N₂ at 0.3 ATM pressure created by vacuum. The reaction rate was faster under vacuum than at 1 ATM (FIG. 2B), suggesting that the low pressure created by the vacuum in the vessel accelerated the reaction.

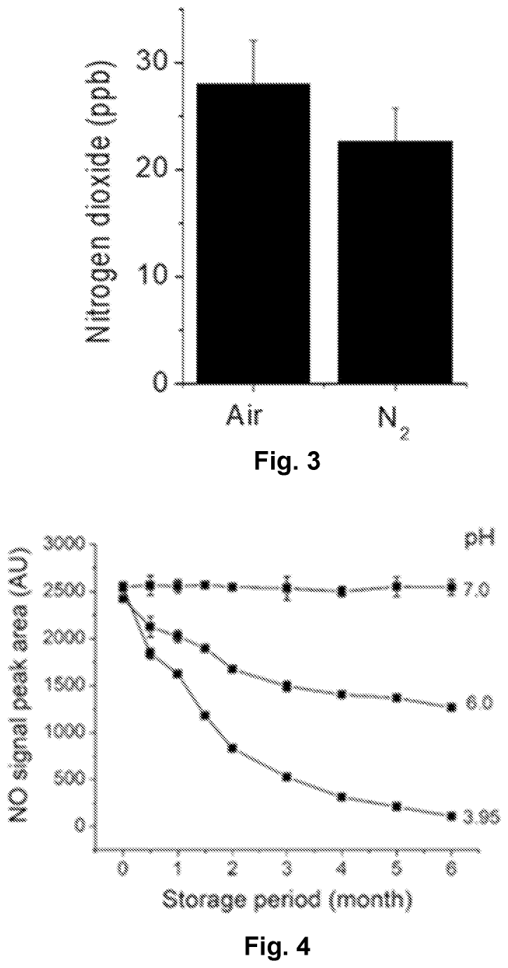

FIG. 3 demonstrates the high purity of the NO generated in the cartridge. The NO₂ levels were less than 30 ppb in 100 ppm NO generated under air and N₂ conditions described above without stirring the sample (i.e., using the cartridge described herein). NO is known to react with O₂ to form NO₂. Previous work indicated that this NO₂ diffuses back into the solution where it is converted to NO and that stirring facilitates this process. In the present disclosure, the sample is not stirred to generate NO. Therefore, we compared NO₂ formation under conditions of air at 1 ATM and N₂ at 1 ATM. There was no significant difference in NO₂ level (mean±S.D, n=3) between air and N₂, indicating that the trace amounts of NO₂ detected were formed while the NO passed through the tubing system to the CAPS NO₂ analyzer for detection of NO₂. Hence, NO₂ formation is negligible during NO generation in the current NO-cartridge system (FIG. 1), even under oxygen conditions.

In the literature, S-nitroso-N-acetylcysteine (SNOAC) is prepared by dissolving NAC in HCl (0.2 to 0.6 M) and mixing it with sodium nitrite solution at a ratio of 1:1. Similarly Cu(I) ion-mediated degradation of S-nitrosothiols has been demonstrated by combining soluble cupric salt with a reducing agent of ascorbic acid or miscible form of cuprous salts. However, there are no reports of generating NO gas in bulk amounts by combining solutions of NAC, nitrite, and Cu(I) ions or reacting this chemical mixture with water as described here. For the first time, we have demonstrated in this disclosure that a highly pure form of NO gas can be generated within a few minutes by reacting water with the mixture of NAC, sodium nitrite, and cuprous chloride crystals. This method can advantageously be used for generating NO rapidly, such as for use in emergency situations. The main observation is that the rate of reaction between these chemicals to generate NO is faster and safer (e.g., containing less NO₂) when water is added to the mixture of these three crystal chemicals rather than mixing the aqueous solution of these chemicals, as previously described in the literature. Seven reactions, shown below, are taking place simultaneously to generate NO. All these reactions occur under acidic conditions provided by a proton donor from the carboxylic group of NAC. Acidified nitrite reacts with NAC to generate SNOAC. Cu(I) degrades SNOAC to NO. Cu(I) ions reduces acidified nitrite to NO. Cu(II) is reduced back to Cu(I) by NAC. The final products are NO and disulfide acetylcysteine.

NAC—COOH→NAC—COO⁻+H⁺

NO₂⁻+2H⁺↔HNO₂+H⁺↔NO⁺—H₂O

NO⁺—H₂O+NACSH→SNOAC+H₂O

NO⁺—H₂O+Cu(I)→NO+Cu(II)+H₂O

SNOAC+Cu(I)→NO+Cu(II)+′SNAC

NACSH+Cu(II)→NACS′+Cu(I)

NACS′+NACS′→NACSSCA

The generation of NO gas during the reaction solution form of these chemicals has been described in the literature. This NO is converted to nitrous acid and nitrite following the reaction with soluble oxygen. The method of combining the crystals of these chemicals and then reacting with water in the absence of O₂ to generate a bulk amount of NO has not been previously described. The rate of this reaction for generation of reaction is very rapid without generating toxic amounts of NO₂ as a byproduct. Therefore, this method represents a great advance in generation of NO for on-demand use in inhaled NO therapy.

In this modified process, 0.23 moles (3.75 g) of N-acetylcysteine (NAC), 0.23 moles (1.59 g) of nitrite, and 0.0045 moles (0.45 g) of Cu(I)Cl are placed in the reaction vessel 110 described above, which is closed with the self-sealing plug connector 114 (FIGS. 1A, 1B). Mixing these three chemicals generates 0.23 moles (500 mL) of NO if all nitrite is converted to gas. The socket coupler 122 is snapped to the reaction plug connector 114, and the water is injected. The reaction takes place rapidly, and the NO generated is transferred into the empty graduated syringe by force of pressure from the exit port 116. The reaction vessel is not shaken or disturbed after the water is injected.

Stability of NO during storage—In a series of experiments, the instability of NO was investigated and a method for increasing stability was discovered. NO undergoes redox interactions with metal ions to form nitrogen oxide species. These redox reactions are highly influenced by the acidic pH of the reaction mixture. Therefore, we investigated the stability of NO that is generated in the reaction vessel to determine how long it can be used for inhaled NO (iNO) therapy. NO content in the fixed volume of generated gas was measured by chemiluminescence assay at varying time intervals for 180 days. The chemiluminescence assay was specific for NO gas only and not for other nitrogen oxide gases such as NO₂, nitrous oxide (N₂O), etc. NO was generated in a septum-sealed reaction vessel at up to 50 psi pressure. Exactly 25 μL of gas was drawn with a gastight Hamilton syringe and then injected into the stream of nitrogen gas (3 L/min) that was drawn into an NO analyzer for measurement of NO gas. The area under the curve of NO signal from the NO analyzer is directly proportional to NO concentration. The initial peak area that was measured within an hour of NO generation was considered to be baseline NO concentration. The change in peak areas of subsequently injected samples at varying time intervals is shown in FIG. 4. The NO concentration in the reaction vessel was stable for 2 days and then decreased with time. Fourier-transform infrared spectroscopy (FTIR) analysis shows that approximately 80% of NO was converted predominantly to N₂O and minor amounts of NO₂ at the end of 180 days. The final pH of the reaction mixture in the reaction vessel was 3.9 after completion of NO generation. The interaction of NO with copper ions under this acidic pH generated N₂O:

2CuCl+2NO+2H⁺+2Cl⁻→2CuCl₂+N₂O+H₂O.

Next, we increased the pH of the reaction mixture from 3.95 to 6 and 7 using a basic disodium hydrogen phosphate (Na₂HPO₄). Any other basic compounds like sodium or potassium hydroxide and other alkaline solutions can also be used. The pH of the reaction mixture was raised from 3.9 to 6.0 and 7.0 by injecting a required amount of Na₂HPO₄ into the reaction vessel after NO generation. The stability of NO in the reaction vessel was measured at varying time intervals for 180 days. As shown in FIG. 4, the stability of NO was increased by increasing the pH of the reaction mixture. At pH 7.0, NO was completely stabilized for 180 days, confirming that the acidic pH of the reaction mixture is responsible for the instability of NO generated in the reaction vessel. The volume of NO did not change throughout this period as measure by a pressure regulator. At pH 7, any unreacted cuprous chloride is insoluble in aqueous solution; therefore, it does not react with NO to form N₂O. Copper ions were precipitated as copper phosphate at the neutral pH. Alternatively, the stability of NO was extended for 3 months by using only the optimum amount of cuprous chloride required to generate NO. In this case, the mole ratio of sodium nitrite, NAC, and Cu(I) is revised from 1:1:0.2 to 1:1:0.02.

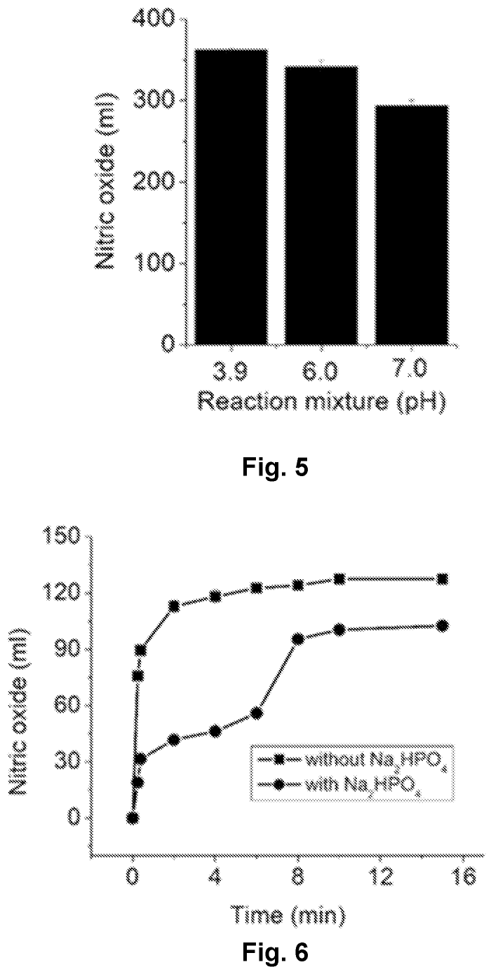

In the aforementioned experiment, the pH was raised by injecting a desired amount of Na₂HPO₄ into the reaction vessel after NO gas generated. However, this procedure is not practical to use in the NO-cartridge because the vessel seals due to the pressure generated by the reaction; therefore, an additional reactant cannot be added to the airtight reaction vessel after generation. One option is to transfer the NO to another container after generation in the reaction vessel, but his would add an extra complication to the NO delivery systems. Instead, we have developed a system to increase the stability of NO in the reaction vessel itself. The reaction between nitrite, NAC, and Cu(I) chemicals to generate NO gas is very fast following the addition of water, even without stirring (FIGS. 2A and 2B). Approximately 80% of NO is generated within the first minute of reaction initiation. Na₂HPO₄ would be expected to dissolve at a slow rate in aqueous solution compared to the rate of NO generation. Thus, we hypothesized that adding Na₂HPO₄ to the mixture of NO-generating chemicals may not significantly affect NO generation. To test the effect, we included Na₂HPO₄ with the nitrite, NAC, and Cu(I) mixture prior to reaction initiation. We used amounts sufficient to raise the pH from 3.9 to 6.0 and 7.0 by the end of the reaction. The mole ratio of nitrite, NAC, and Cu(I) was modified for this experiment from the original ratio of 1:1:0.2 to 1:1.25:0.2. The amount of Na₂HPO₄ required to raise the pH of the reaction mixture from 3.9 to 6.0 in one trial and to 7.0 in a second trial was added to the reaction vessel first, and then thoroughly mixed nitrite-NAC-Cu(I) chemicals were transferred. The typical NO-generating mixture consists of 1.17 g sodium nitrite, 3.45 g NAC, and 0.33 g Cu(I)Cl. To this reaction mixture, 2.5 g of Na₂HPO₄ was added to adjust the pH to 6.0, and 3.0 g Na₂HPO₄ was added to adjust the pH 7.0. The atmospheric air in the reaction vessel was replaced with nitrogen gas as described above. The reaction was initiated by injecting an appropriate amount of water, and the volume of NO generated was measured. As shown in FIG. 5, 362 mL of NO gas was generated at pH 3.9 in the absence of Na₂HPO₄, 342 mL was generated in the presence of 2.5 g Na₂HPO₄ (for pH 6.0), and 293 mL of NO gas was produced in the presence of 3.0 g Na₂HPO₄ (for the pH 7.0). Approximately 20% less NO was generated at pH 7.0 than without the addition of the Na₂HPO₄, however the purity was not affected by the addition. 100% of the NO was stable for 180 days (FIG. 4). Therefore, the amount of Na₂HPO₄ required to maintain the pH at 7.0 can be used to prevent decay of NO in the cartridge. The mole ratio of sodium nitrite, NAC, Cu(I)Cl, and Na₂HPO₄ is 1:1.25:0.2:1.25 for the final pH of 7.0. Although inclusion of the alkaline compound in the cartridge results in the generation of 20% less NO, this loss can be compensated for by increasing the reactants by 20%. It may also be possible to use slightly larger crystals of Na₂HPO₄ to delay the solubility for several minutes, which would increase the yield of NO to 100% . . . . Thus, FIG. 5 shows that the inclusion of Na₂HPO₄ c into the NO-generating chemical mixture raises the pH, but partially decreases the amount of NO generated. However, the stability of the NO is maintained for at least 180 days.

It is important to know the rate of NO generation in the cartridge to assess how quickly it can be delivered to patients. As discussed above, a pH of 7.0 provides stability of NO in the reaction vessel. The reaction of nitrite with NAC to generate SNOAC and the reduction of nitrite to NO by Cu(I) are largely dependent on the acidity of the reaction mixture. Inclusion of Na₂HPO₄ in the reaction mixture raises the pH and slows the rate of NO generation, as shown in FIG. 6. Hence, we investigated the rate of NO generation at the concentration of Na₂HPO₄ required to maintain pH 7.0. The combined mixture of sodium nitrite, NAC, Cu(I)Cl, and Na₂HPO₄ at the mole ratio of 1:1.25:0.2:1.25 was used to generate NO in the reaction vessel. In the absence of Na₂HPO₄ (FIG. 6, -▪-), the initial rate of NO generation was fast and reached the maximum amount by 5 minutes after reaction initiation. In the presence of Na₂HPO₄ (FIG. 6, -•-), NO was generated in three phases and reached a maximum amount after 15 minutes. The initial rapid phase appeared to be direct reduction of nitrite to NO by Cu(I), in the slow second phase, acidified nitrite reacted with NAC to generate SNOAC, and in the fast third phase, SNOAC was degraded to NO by Cu(I) ions. This rate of NO generation was influenced by the rate of Na₂HPO₄ dissolution in the aqueous reaction mixture.

Since approximately 80% of the NO is generated within approximately one minute in the absence of Na₂HPO₄, NO gas generated without the Na₂HPO₄ is available for therapy within minutes of reaction initiation. However, the shelf-life of the cartridge after generation of NO is less than 48 hours. It can be used when NO intervention is needed quickly and for a short duration of treatment (<48 hours). Advantageously, in the presence of Na₂HPO₄, the NO cartridge can have a long shelf-life for a minimum of 6 months or more. FTIR studies investigating longer-term storage will be performed to establish the destabilization rates to prevent unwanted degradation resulting in NO₂ and N₂O.

We hypothesized that under appropriate conditions, the dry chemicals in the cartridge and the NO yield in the cartridge should remain stable for one to two years prior to reaction with water. The purpose of this experiment was to find conditions that increase the stability of chemicals and shelf-life of the cartridge. NAC, nitrite, and Cu(I)Cl are slightly hygroscopic at atmospheric humidity and temperature. NAC degrades to diacetylcysteine disulfide, nitrite oxidizes to nitrate, and Cu(I) oxidizes to Cu(II) over time in the presence of oxygen and moisture. This instability is accelerated when they are mixed together because intermediate products are formed, including SNOAC, NO, nitrogen dioxide, and Cu(II)Cl₂. Completely eliminating the moisture content that is absorbed into chemicals as well as outside moisture could increase the compatibility and stability of this chemical mixture. The moisture content in the chemicals is stripped by exposing them to heat without affecting the chemical nature of the compounds and stored them in a desiccator. The moisture content was found to be 0.3% in NAC, 0.2% in sodium nitrite, and 0.25% in Cu(I)Cl. We are currently assessing the long-term stability of the dry mixture of NAC, sodium nitrite, and Cu(I)Cl under air, air evacuation (70%), nitrogen, and nitrogen evacuation in airtight sealed glass vials by measuring the amount of NO generation at 6-month interval for 2 years. Chemicals were stored at the ratio of 1:1:0.2 under the indicated conditions in sealed vials. The amount of NO generation was measured following the reaction with water. Values are expressed as mean±SD. N=3. The values are expressed as a percentage of baseline NO concentration. FIG. 7 shows the results after the first 6 months under N₂ and O₂ at 1 ATM pressure and 0.3 ATM pressure in gastight sealed tubes. The chemical mixture that was stored under the moisture-free nitrogen gas generated 100% NO gas. NO generation decreased by approximately 11% in samples stored under atmospheric air at 1 ATM and by approximately 5% in samples stored at 0.3 ATM. Samples stored in air had some moisture and O₂. Chemicals in these samples tended to lose stability and generate less NO. Even with very little moisture, nitrite can be acidified to HNO₂ in the presence of NAC, and then HNO₂ reacts with NAC thiols to generate SNOAC, which is degraded to NO by Cu(I). This NO reacts with O₂ to form NO₂ and then converts back to nitrous acid. This cycle continues until all O₂ is consumed. The data show that the chemical mixture of NAC, nitrite, and Cu(I)Cl is stable under nitrogen in a sealed container for 6 months. This study will continue for another 18 months under the same conditions.

In a separate experiment, we included moisture-absorbent calcium sulfate crystals (WA Hammond Drierite Co.) in the chemical mixture to study the stability of chemicals under atmospheric air. Calcium sulfate did not interact with NO-generating chemicals in crystal or solution state. If use of calcium sulfate works in the long-term, the cartridge will not need to be flushed with N₂ before evacuation. It is anticipated that the calcium sulfate would not interact with Na₂HPO₄, allowing the shelf-stable NO generation to be performed without N₂ flushing.

Table 1 shows the storage stability of the NO generating chemical mixture of sodium nitrite, NAC and Cu(I)Cl in the presence and absence of base Na₂HPO₄ under nitrogen in gas-tight vials. In the previous experiment (FIG. 7), NO generating mixture was shown to be unstable in the presence of atmospheric air, but it is stable under nitrogen at 1 ATM or 0.3 ATM pressure. As discussed above, Na₂HPO₄ raises the pH and increases the stability of NO after generation in the cartridge, however the effect of Na₂HPO₄ on the stability of the mixture prior to generation was not known. The stability of the four-chemical mixture was investigated under the nitrogen only. The moisture content on the Na₂HPO₄ is 0.5%. Nitrite, NAC, Cu(I)Cl and Na₂HPO₄ mixture was assessed under the moisture-free nitrogen at 1 ATM pressure. We mixed the dry chemicals of 0.345 g NAC, 0.117 g sodium nitrite, 0.33 g Cu(I)Cl, and 0.3 g Na₂HPO₄ and then transferred them to airtight septum-sealed reaction vials in triplicate. The same chemical mixture but without Na₂HPO₄ was stored in another set of vials. The atmospheric air in the vials was replaced with nitrogen gas by flushing and the vials were stored at room temperature. We then measured the volume of NO generated after addition of 2 ml of water at 3-month intervals for 2 years. Results of the first 6 months of storage are shown in Table 1. NO generation was 23% less in the presence of Na₂HPO₄ than in the absence of Na₂HPO₄, consistent with the initially generated amounts described in the previous stability experiment. Baseline NO volumes were considered 100%. Subsequently measured NO volumes are expressed as a percentage change from baseline.

TABLE 1
Stability of the NO generating chemical mixture
in the reaction vessel under the nitrogen.

| Storage | NO₂⁻ + NAC + | | NO₂⁻ + NAC + | |
|---|---|---|---|---|
| duration | Cu(I) | | Cu(I) + Na₂HPO₄ | |
| (months) | NO (mL) | % | NO (mL) | % |
| 0 | 36.5 ± 1.2 | 100% | 28.2 ± 1.5 | 100 |
| 3 | 36.1 ± 0.8 | 98.9% | 28.6 ± 0.75 | 100 |
| 6 | 36.8_± 1.1 | 100.8 | 27.6 ± 1.2 | 97.87 |

The data show that the chemical mixture of NAC, nitrite, Cu(I)Cl and Na₂HPO₄ is stable under nitrogen in a sealed container for 6 months, with a slight reduction of NO at 6 months. This study will continue for another 18 months under the same conditions.

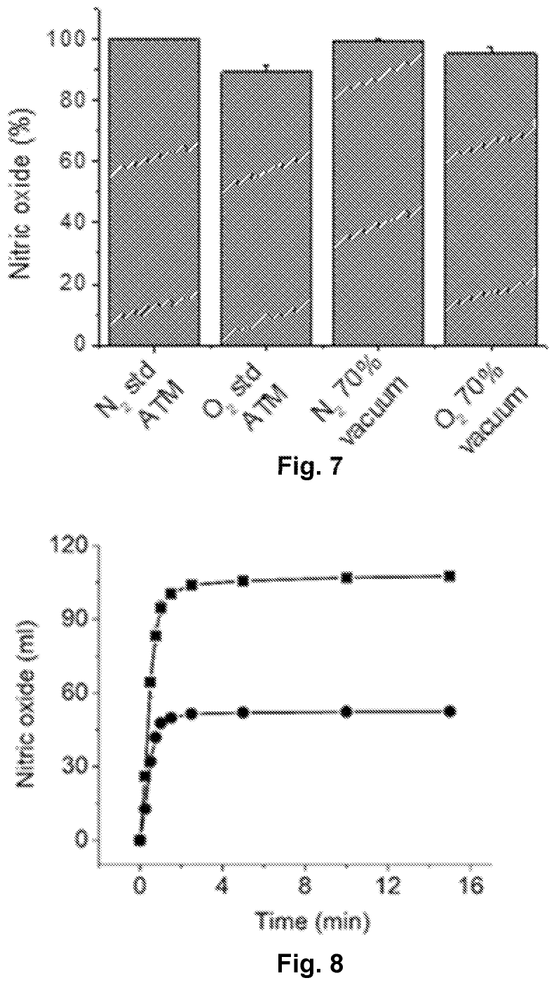

FIG. 8 shows the conversion of nitrite to NO by a combination of ascorbic acid and Cu(II) ions. In short, while ascorbate converts nitrite to NO, the acidity creates an unstable reaction where the nitrite: NO ratio is about 2:1 or about-3:1. By including a small amount of Cu(II) in the reaction, a nitrite: NO ratio of 1:1 can be achieved due to ascorbic acid reduction of Cu(II) to Cu(I).

Ascorbic acid (RH) readily loses hydrogen ion (H⁺ ions) forming ascorbate anion. Hydrogen ions acidify the nitrite to nitrous acid, which has a nitrosonium ion character. Ascorbate anion readily reduces this nitrous acid to NO. Ascorbate anion also reduces Cu(II) to Cu(I). Cu(I) ions reduce nitrous acid to NO rapidly. Cu(II)/Cu(I) undergoes redox cycles as long as ascorbic acid is available. Both the ascorbate anions and Cu(I) convert nitrite to NO under acidic conditions. We used these reactions to make a bulk amount of NO gas. Addition of water to the mixture of 5 mmoles (0.345 g) sodium nitrite, 5 mmoles (0.881 g) ascorbic acid and 0.25 (0.0336 g) mmole Cu(II)Cl₂ at the ratio of 1:1:0.05 under nitrogen in an airtight reaction vessel resulted in rapid generation of NO. As shown in FIG. 8, about 90% of nitrite was converted to NO within a minute. About 98% of nitrite is converted to NO gas in 15 minutes (-▪-). Values are mean±SD, N=3. One nmole nitrite (69 mg) produced 0.98 nmole NO gas (21.56 ml). About 50% of NO gas is produced in the absence of Cu(II)Cl₂ (-•-). The following reactions are taking place during the reaction of sodium nitrite, ascorbic acid (RH) and Cu(II)Cl₂ in aqueous solution to generate NO gas:

RH→R⁻+H⁺

NO₂⁻+2H⁺↔HNO₂+H⁺↔NO⁺—H₂O

NO⁺—H₂O+R⁻→NO+R′+H₂O

RH+Cu(II)→R′+Cu(I)

NO⁺—H₂O+Cu(I)→NO+Cu(II)+H₂O

R′+R′═R—R

Any compounds that are an electron and proton donors (H⁺) can be used in place of ascorbic acid to convert nitrite to NO. Any electron donor compounds (reducing agents) in the presence of low pH conditions can be used in the place of ascorbic acid to convert nitrite to a bulk amount of NO. Cu(I) salts under acidic conditions also converts nitrite to NO to make bulk amount of NO gas. Any other metal ions that undergo redox cycling in the presence of reducing agents under acidic conditions can be used in place of Cu(I) or Cu(II) salts to convert nitrite to NO in bulk amounts.

An additional experiment was undertaken to increase the stability of NO in the reaction vessel generated by nitrite-ascorbic acid and Cu(II) CI. NO that is generated in the reaction vessel is unstable. It slowly converts to nitrous oxide with time under the acidic conditions of the reaction mixture. Therefore, the pH of the reaction mixture was raised from 5.4 to 7.0 by adding a basic solution of Na₂HPO₄ after generation of NO using an infusion pump into the reaction vessel via the quick connectors shown in FIGS. 1A and 1B. NO was quite stable at the neutral pH. Inclusion of Na₂HPO₄ to raise the pH to 7.0 in the nitrite with the ascorbic acid and Cu(II)Cl₂ mixture in the cartridge resulted in a decrease of NO generation from 100 to 75% following the addition of water. However, the rate of NO generation was not affected significantly in the presence of Na₂HPO₄. The typical ratio of nitrite, ascorbate, Cu(II) CI and Na₂HPO₄ of 1:1:0.005:1.25 was used to generate and increase the stability of NO gas in the reaction vessel. The generated NO is stable for 3 months in the reaction vessel. This is an alternative procedure for nitrite-NAC-Cu(I)—Na₂HPO₄ mixture to generate NO in the NO-cartridge.

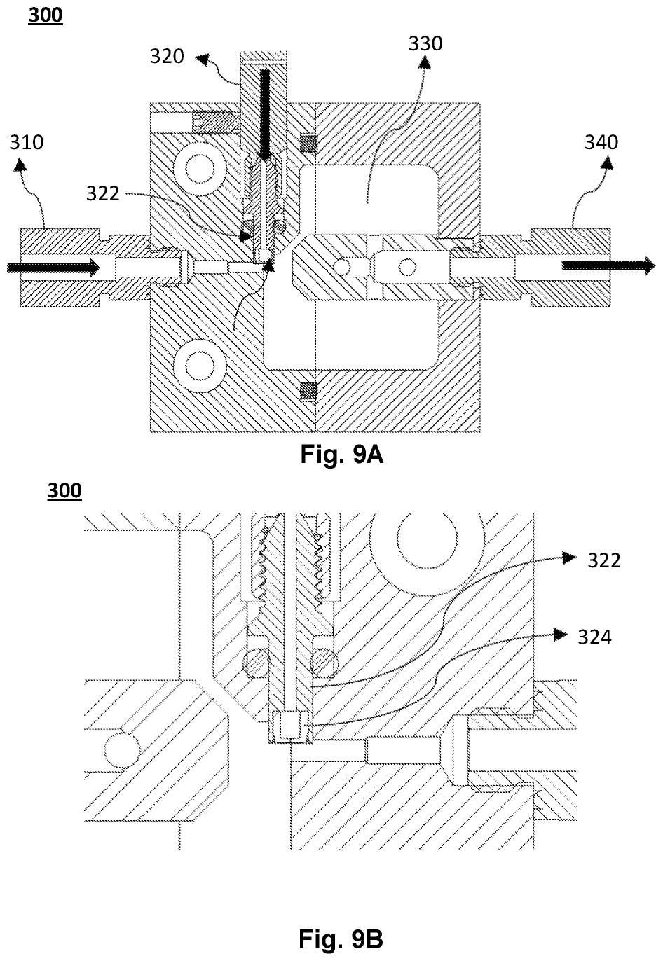

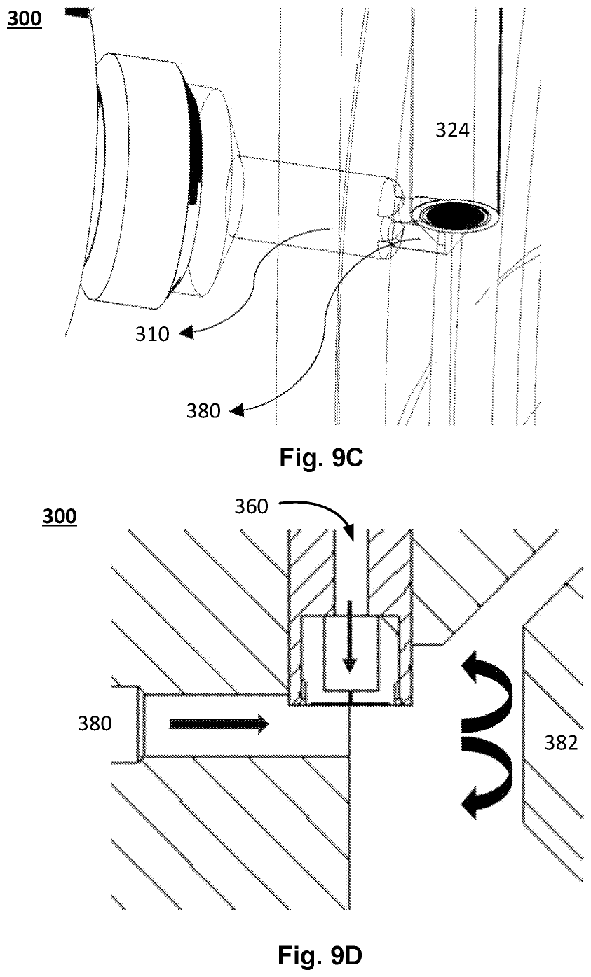

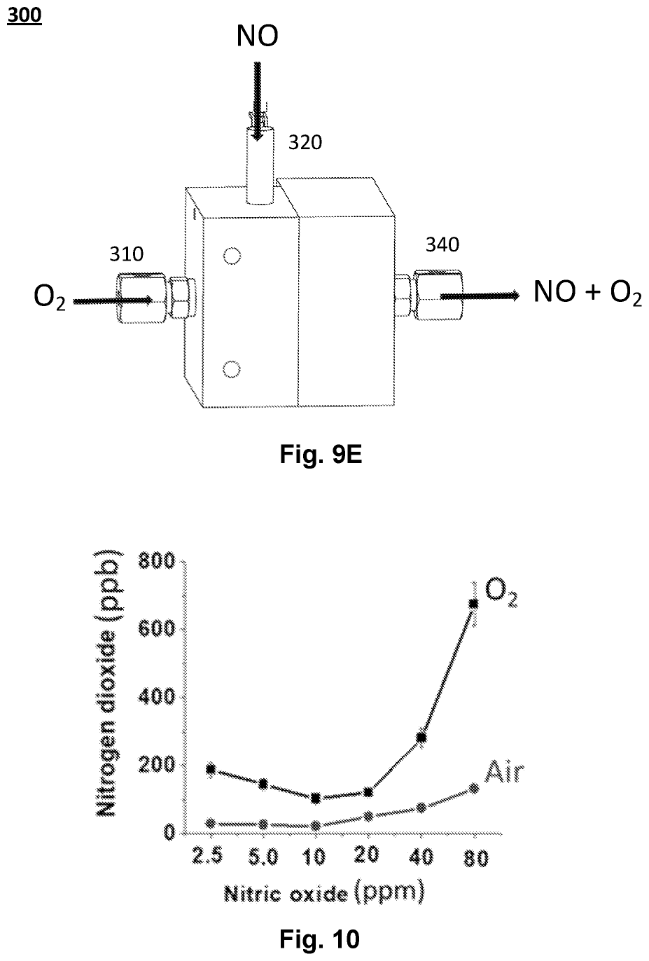

Mixing the generated NO with carrier gas: FIGS. 9A-9D are diagrams of the mixing chamber 300 and its assembly that is used for rapid mixing of the generated NO with carrier gas (e.g., N₂, O₂ or air). Mixing the concentrated NO with the carrier gas to dilute it in the mixing chamber prior to delivery to the patient minimizes the NO₂ formation in the iNO system. The mixing chamber allows for the successful infusion of NO into the carrier gas. The mixing chamber (shown in a particular embodiment having a height 1.6″ and width 1.5″) consists of four primary components: the carrier gas inlet 310, the NO inlet 320, mixing space 330 (e.g. the empty space for the carrier gas and NO to mix), and combined gas outlet 340 (FIG. 9A). The size, placement, and geometry of all primary features were chosen to reduce the amount of NO₂ created as result of NO reaction with O₂ in the chamber. In an embodiment, the mixing chamber 300 is constructed from two halves of a stainless-steel block fastened together to form an internal cavity that defines a 20 ml mixing space 330 of gases.

In the depicted embodiment, an O-ring seals the two halves and ⅛″ OD tube compression fittings attach to the carrier gas inlet 310 and combined gas outlet 340 ports. The NO inlet 320 extends into the mixing chamber 300 via nozzle assembly that includes a 13 Birmingham Wire Gauge (BWG) hypodermic stainless-steel tube 322. The tube 322 is capped with a stainless-steel, press-in, insert with a nozzle 324 (FIG. 9B). The nozzle orifice can be a ruby (corundum) orifice. In the depicted embodiment, the NO nozzle 324 has a fixed outside diameter of 0.06″, and thickness of 0.01″ and varying internal diameter (ID) orifice ranges from 0.0001″ (2.54 μm) through 0.035″ (889 μm). The nozzle is different from flow restrictors. The NO₂ formation was previously measured at varying nozzle orifice sizes ranging from 10 μm to 889 μm. The lowest NO₂ formation was observed at 10 μm at a manageable level of pressure across the NO gas-line. NO nozzle 324 orifice sizes 0.0003937″ (10 μm)—and 0.000984″ (25 μm) were used for the benchtop prototype. This small orifice not only reduces the NO₂ formation but also to controls the infusion of microliters of NO into the carrier gas. The two holes shown in the drawing are optional mounting holes.

The carrier gas inlet 310 brings the carrier gas into the mixing chamber 300 to intersect with the end of the NO inlet 320 tube. The geometry of the passageway 320 is such that the 1/16″ ID channel cross-sectional area is reduced by 40% into a slot channel that is perpendicular to the ruby orifice surface (FIG. 9C). The reduction in cross-section 380 is intended to cause a localized increase in the carrier gas velocity as it crosses the surface of the orifice. The carrier gas velocity and interaction with the NO gas flow are used to establish a rapid mixing and dilution of the NO to avoid NO₂. The mechanism by which NO₂ gas is generated when combined with the carrier gas is highly dependent on the local concentration of NO. Therefore, the mixing chamber features were designed to move NO away from the inlet tube 320 orifice as quickly as possible and rapidly dilute the NO into the carrier gas. The mixing space 330 widens immediately after the mixing point to induce turbulent flow of the gas mixture. An obstruction 382 is placed shortly after the mixing point to further promote turbulent flow and disperse the gas into localized eddies (shown by curved arrows) (FIG. 9D). Turbulent flow continues throughout the rest of the mixing chamber as the gas makes its way through the convoluted exit path. The abrupt change in cross section induces turbulent flow to disrupt the NO stream and mix more evenly into the carrier gas. The obstruction prevents the gas from moving directly from the inlet to the outlet. The gas stream must bend and curve to enter the obstruction through holes cross drilled in the obstruction before being forced out of gas outlet 340, ensuring the gases are thoroughly mixed before exiting the chamber. The mixing chamber architecture increases the flow velocity of NO as well as O₂ to mix them rapidly to minimize the dilution time of NO with O₂ that minimizes the NO₂ formation. Other obstruction shapes can be envisioned by one of ordinary skill in the art. FIG. 9E is a diagram of the external mixing chamber 300 showing the gas flow.

FIG. 10 shows the NO₂ formation during the mixing of NO with O₂ in the mixing chamber 300. As mentioned above, toxic NO₂ gas is formed as a result of NO reaction with oxygen. While NO₂ generation during the delivery of NO is unavoidable, it can be minimized to safe levels. There are three sources of NO₂ generation. The first is during the synthesis of NO, the second during the dilution of NO with O₂, and the third during the delivery of NO to patients. The efficacy of inhaled NO delivery can be improved by minimizing the co-delivery of NO₂. NO₂ formation during the synthesis is in trace levels of about 25 ppb. In the present mixing chamber, a NO nozzle orifice 324 size of 25-μm was used. An improved-sensitivity NO₂ analyzer has also been included in the system. NO₂ is measured using a highly sensitive (1 ppb) and reliable CAPS NO₂ analyzer. Simultaneously, NO is also measured by chemilumiscence assay. FIG. 10 shows the NO₂ formation during the dilution of NO in carrier gas of air (-•-) or 100% oxygen (-▪-) at therapeutic doses. Values are mean±SD, n=4. The therapeutic dose of NO is in the range of 2 to 80 ppm with a mean dose of 20 ppm. The percentage of oxygen in the inspired gas is in the range of 21% to 100%. The NO₂ level is <1 ppm at the maximum dose of NO (80 ppm) in 100% O₂. This level is less than the FDA safety limit of 1 ppm. The NO₂ level of 43 ppb in air as a carrier gas (21% O₂) at 20 ppm NO is close to environmental safety limitation of <50 ppb. (Table 2).

In order to test the NO generated by the reaction of nitrite, NAC and Cu(I)Cl with water in the reaction vessel 110, the NO was transferred into mini-canisters and shipped to CONSCI, Ltd., which analyzed the total gas composition by the FTIR method. The CO₂ levels were miniscule. A small percent of nontoxic NO₂ (0.14%) was detected and likely formed during the transfer of NO gas into the canisters. Analysis by a CAPS NO₂ analyzer (Aerodyne Research, Billerica, MA) showed that NO₂ was 0.26% (26 ppb in 100 ppm NO) in NO collected from the air-evacuated reaction vessel. Thus, traces of NO₂ formed during the handling of sample for analysis. These results confirm the synthesis of 99.9% pure NO using the cartridge. No further purification is required as it is with the technology of other companies.

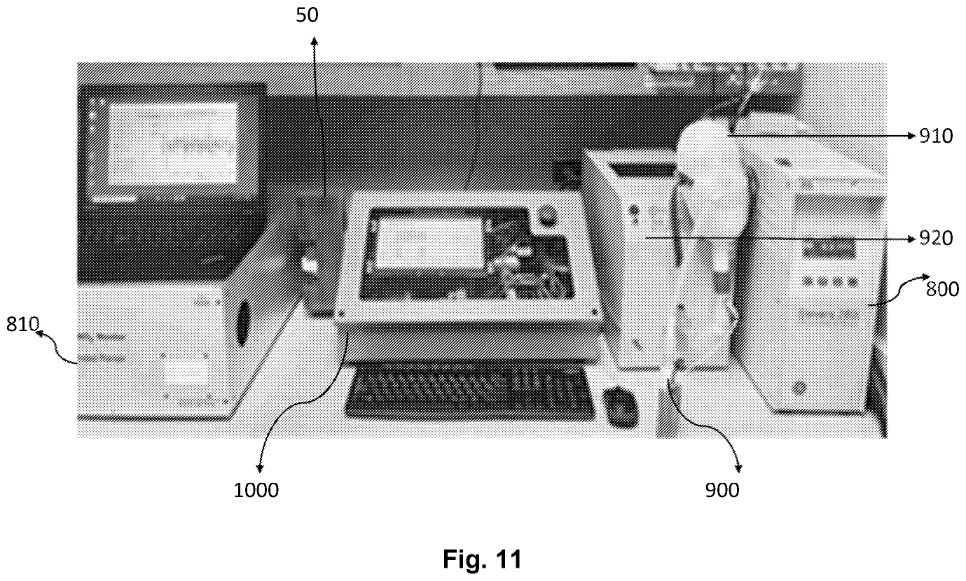

FIG. 11 illustrates an embodiment of a benchtop iNO delivery system that simulates a patient NO delivery system and is used to determine the NO and NO₂ levels in the therapeutic inhaled NO gas before and after (trachea) inhalation. NO that is generated and diluted in carrier gas to therapeutic doses needs to be delivered to patients. NO₂ generation in the breathing circuit is dependent on the prescribed dose of NO, the O₂ percentage in inspired air, and the flow rate of the inspired gas. All these factors influence the NO₂ generation. Hence, the benchtop device that simulates a patient NO delivery system is used to investigate the formation of NO₂ from the mixing point of NO with O₂ in the device to lungs. We applied varying NO doses, flow rates and O₂ saturation. The setup for the benchtop NO delivery system is shown in FIG. 11. It consists of a prototype device 1000 (which includes a mixing chamber 300 as described above) associated with an NO cartridge 50 (a previous model from cartridge 100 disclosed herein) that was built with computer program-controlled NO generation and mixing of NO with O₂. It also includes a spontaneous breathing ASL 5000 simulator (a lung model) 920, an airway upper respiratory system model 910 that simulates a 25-year-old, 75-kg person, and a nasal cannula/high-flow nasal cannula as a non-invasive interface system 900, an NO analyzer 800, and NO₂ analyzer 810. The therapeutic gas (NO+O₂) exit line from the benchtop iNO device is connected to the proximal side of the nasal cannula. The nasal upper airway model 910 is connected to the tracheal tube, which is then attached to the ASL 5000. A trap to prevent the reentry of NO and NO₂ gas in exhaled air is placed at the tracheal tube just before the ASL 5000. Three-way ports are placed at the beginning of the inspiratory breathing circuit and at the trachea for gas sampling. The ends of these ports are connected to 3-way stopcocks, which are connected to the NO analyzer and NO₂ analyzer. The ASL 5000 is initiated at the normal healthy person settings (breathing frequency=15 breaths/min, Rᵢₙ=4 cm H₂O/L/s, Rₒᵤₜ=4 cm H₂OL/s, C=60 mL/cm H₂O, tidal volume (VT)=685 mL, Pₘₐₓ=11.95 cm H₂O, increase=33%, and release=28%; at varying inspired gas flow rates. We measured the baseline NO levels by NO analyzer and NO₂ analyzer simultaneously at the preset (prior to inhalation) and trachea level. Gas flow and pressure in the proximal side nasal cannula by pneumotachometer and within the respiratory system by ASL 5000 also was measured (FIG. 11). Table 2 shows NO and NO₂ levels at the prior of inhalation and at trachea level.

TABLE 2
NO and NO₂ levels were measured prior to inhalation (pre-set) and at the trachea during
inhalation of 20 or 40 ppm NO at a flow rate of 10, 20, or 30 L of air or 100% O₂.

| | Nitrogen | | | | Air (21% O₂) | | | | Oxygen (100%) | | | |
|---|---|---|---|---|---|---|---|---|---|---|---|---|
| | Pre-set | | Post-set | | Pre-set | | Post-set | | Pre-set | | Post-set | |
| Flow | NO | NO₂ | NO | NO₂ | NO | NO₂ | NO | NO₂ | NO | NO₂ | NO | NO₂ |
| rate | (ppm) | (ppb) | (ppm) | (ppb) | (ppm) | (ppb) | (ppm) | (ppb) | (ppm) | (ppb) | (ppm) | (ppb) |
| 10 L/min | 20 | 22 ± 7 | 11 ± 6 | 32 ± 5 | 20 | 70 ± 18 | 11 ± 4 | 40 ± 10 | 20.5 | 192 ± 25 | 12 ± 4 | 105 ± 21 |
| 20 L/min | 20 | 21 ± 8 | 14 ± 5 | 28 ± 12 | 20 | 65 ± 12 | 13 ± 5 | 38 ± 6 | 20.6 | 136 ± 31 | 14 ± 6 | 97 ± 17 |
| 30 L/min | 20 | 19 ± 5 | 18 ± 8 | 26 ± 8 | 20 | 43 ± 15 | 17.5 ± 6 | 34 ± 5 | 20.4 | 93 ± 16 | 17 ± 5 | 78 ± 9 |
| 10 L/min | 40 | 32 ± 12 | 23 ± 11 | 65 ± 18 | 40 | 164 ± 26 | 26 ± 6 | 132 ± 31 | 42.6 | 550 ± 65 | 24 ± 8 | 290 ± 35 |
| 20 L/min | 40 | 35 ± 15 | 28 ± 9 | 45 ± 12 | 40 | 108 ± 22 | 28 ± 11 | 110 ± 16 | 41.5 | 481 ± 34 | 27 ± 9 | 263 ± 19 |
| 30 L/min | 40 | 28 ± 9 | 38 ± 15 | 36 ± 8 | 40 | 77 ± 25 | 35 ± 7 | 105 ± 13 | 38.5 | 325 ± 18 | 35 ± 5 | 475 ± 25 |

Baseline NO₂ formation was established using inert nitrogen gas as the NO carrier. The baseline NO₂ levels before the pre-set at the NO exiting point from the device were less than 25 ppb. However, the post-set (tracheal) NO₂ levels are an indicators of NO₂ formed at the interface of nasal/facemask from the entrance of room air into the upper respiratory system in addition to the pre-set level. These NO₂ levels are highly variable, as the interface between air mixing and inspired gas residence time is highly dependent on the flow rates of carrier gas and breathing mechanics. The post-set NO₂ levels were higher than pre-set levels at these settings and needed a detailed investigation. Table 2 shows NO and NO₂ levels at pre-set and post-set for two different NO doses (20 and 40 ppm) and varying carrier gas flow rates. NO₂ generation was less than 0.17 ppm when air (21% O₂) was used as the carrier gas and 0.5 ppm when 100% oxygen was used as the carrier gas. These values were less than the safety limit (1 ppm) set by the FDA for patient populations. The post-set NO values were significantly less than pre-set values and highly dependent on flow rates of inspired gas. The post-set NO₂ values were highly dependent on the O₂ saturation and flow rates of inspired gas, indicating that all these factors need to be considered in iNO therapies.

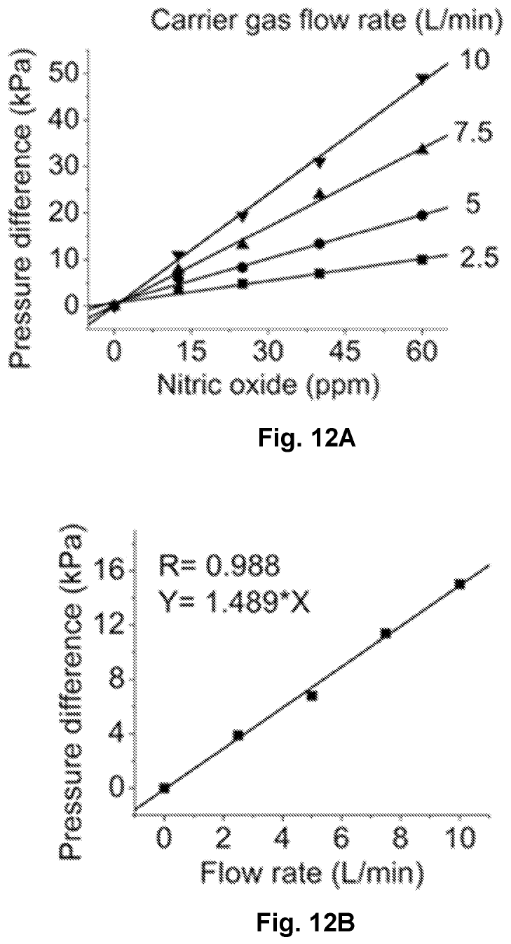

FIGS. 12A and 12B show the correlation of gas pressure difference between manifold (NO gas-line 320) and mixing chamber 300 (carrier gas) with NO dose. In our earlier disclosure, NO was transferred from an NO-cartridge to a syringe reservoir and then dispensed into a mixing chamber to dilute with O₂ via a NO gas line (manifold). The gas pressure sensors were placed in the manifold and in the mixing chamber. The manifold pressure indicates NO pressure and mixing chamber pressure indicated the carrier gas pressure. The manifold NO pressure must be higher than the mixing chamber O₂ pressure for NO to be injected into O₂. Studies for the present system were performed using the earlier device described in (PCT/US2021/071242,) in which the original 25-μm nozzle was replaced with a 10-μm nozzle. The NO pressure in the NO-manifold and carrier gas pressure in the mixing chamber were measured at various doses of NO (12.5 to 60 ppm) with various flow rates of carrier gas (2.5 to 10 L/min). FIG. 12A shows the comparison of the NO dose with the pressure difference (NO pressure with respect the carrier gas pressure). Pressure difference was proportionally increased with increasing concentration of NO and carrier gas flow rates (FIG. 12A). The pressure difference for 20 ppm NO from 2.5 to 10 L/min flow rates was obtained from the linear regression analysis. These pressure differences were plotted against carrier gas flow rates. This pressure difference was positively correlated with the carrier flow rates (FIG. 12B). Based on the slope, the pressure difference for 20 ppm NO was 1.489 kPa per 1 L/min flow rate. These data can be used to develop the algorithm for the software program. In this case, the syringe NO reservoir and syringe pump are not required to maintain the pressure difference on the manifold. Hence, in system disclosed herein, a pressure regulator is used to maintain the desired pressure in place of an NO reservoir and syringe pump. This device would be simple, small, and lightweight for use including in ambulatory patients.

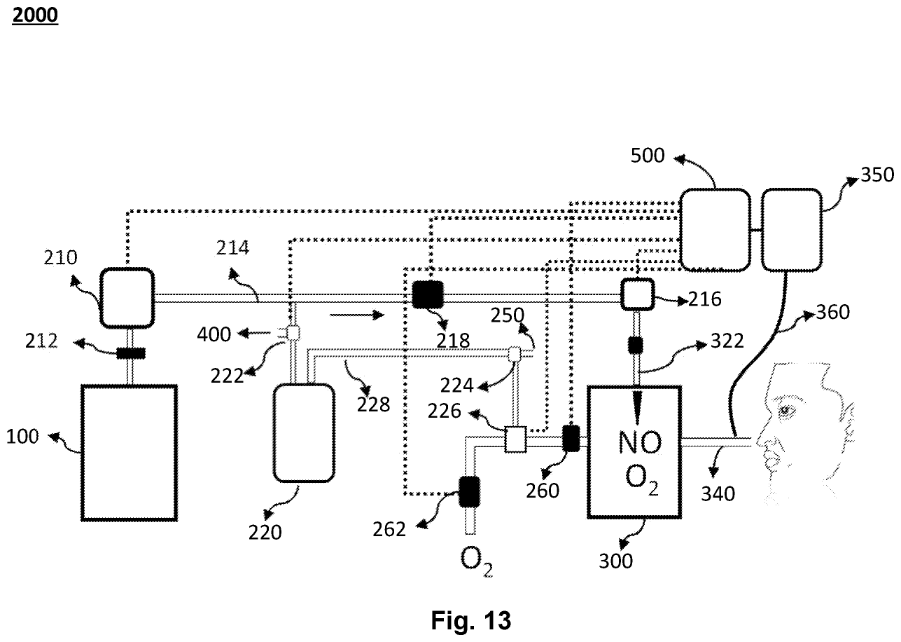

Unlike other systems where the NO dosage is controlled primarily by changing the concentration of NO provided to the system, the NO dosage provided by the current NO delivery system is based on the pressure difference between NO gas and inspired oxygen. The pressure difference between NO and carrier gas governs the NO dosing and is achieved by the nozzle system (NO inlet 320, nozzle tube 322, and NO delivery nozzle 324), where the nozzle 324 size can be changed according to need. FIG. 13 is a schematic diagram of an example pressure difference-based iNO device 2000, which can be used in both portable and bench-top systems. The complex hardware system in our previous system, used to combine the reactants to initiate the reaction for generating NO, has been replaced with the revised cartridge 100 (see FIG. 1). A closed-loop electronic, gas regulator 210 (Clippard) was introduced to control and dosing of NO from the pressurized cartridge 100 to the mixing chamber 300. The moisture trap system 212 comprising calcium sulfate crystals is placed between cartridge 100 and gas regulator 210. The gas exit line 214 is connected to an isolation valve 216. The outlet of gas line 214 is connected to a mixing chamber 300 through the nozzle assembly 320, where nozzle 324 is a 10 or 25 μm orifice ruby straight hole type (Bird Precision, Waltham, MA). Pressure sensor 218 is placed on the gas exit line 214 (also referred to as an NO-manifold). The straight arrow indicates the direction of gas flow. A motor pump 220 is placed to use as a vacuum pump to remove gas from the gas exit line 214 to an exhaust system 250 or as an air pump to use atmospheric air 400 as the carrier gas 228 by way of 3-way solenoid valves 222, 224, 226. The gas sensor 260 is placed at the carrier gas entry of mixing chamber 300. The carrier gas (inspired oxygen) is connected to mixing chamber 300 via gas flow regulator 262. The exit line 340 through which the combination of NO and O₂ from the mixing chamber is connected to the nasal cannula ventilation. The gas sampling line 360 to measure NO, NO₂, and O₂ is connected to the gas sensor system 350. The dotted lines represent connections of parts to a central processing system 500. The maximum limit of carrier gas flow rate for the illustrated device is 10 L/min, however the mixing chamber 300 can be increased in size if a higher carrier gas flow rate is desired. The device, when integrated with miniature air pump 220, can be used as a source of carrier gas outside of hospitals in the absence of compressed cylinders. Because the NO-cartridge system 100 has been simplified to eliminate the previously-described NO reservoir and syringe pump, the weight of the device is substantially lower than previous devices, allowing it to be used as a portable system. Advantageously, this system can be used in and out of hospitals without compressed oxygen cylinders.

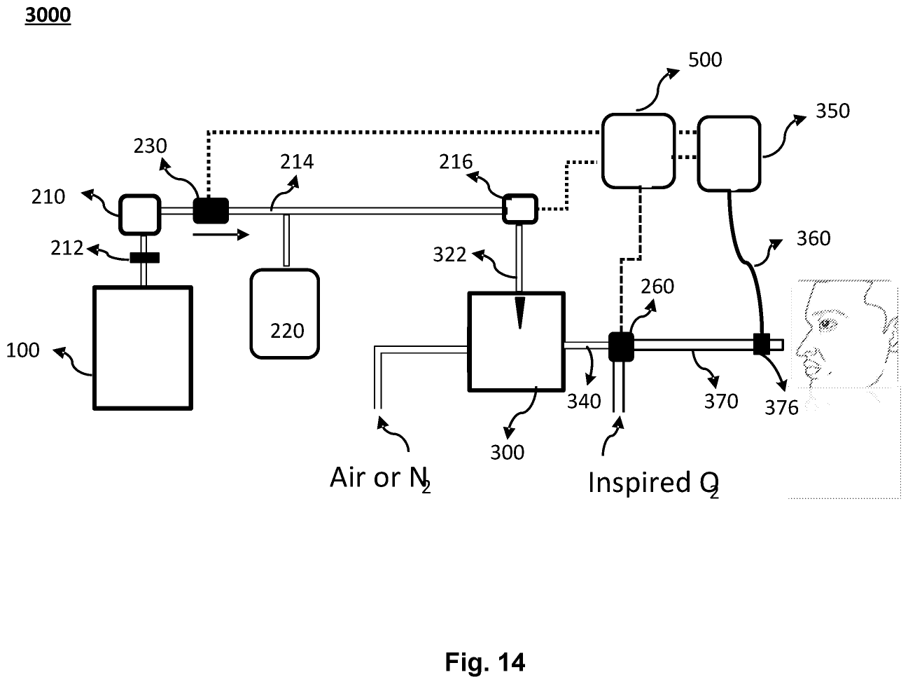

FIG. 14 is a schematic diagram of an example iNO system 3000 that can be used in clinical settings such as hospitals. In this device, the NO delivery to the patient in the mixed gas is based on the pressure difference between the NO and carrier gas (see discussion related to FIGS. 12A and 12B). This pressure difference ensures that the optimal NO amount is delivered in the carrier gas without the formation of toxic amounts of NO₂ when the NO combines with O₂ in the carrier gas. To govern the pressure difference, the NO reservoir and infusion pump from our previous system have been replaced with a programmatically controllable pressure regulator 218. The NO-cartridge 100 that is placed in receptacle of the system 3000 is in fluid communication with the carrier gas in the mixing chamber 300. This fluid communication via the stage 1 fixed gas pressure regulator 210, the stage 2 gas pressure regulator 218 (closed-loop electronic regulator from Clippard), an isolation valve 216 (The Lee Company) and nozzle assembly 320 (including a 10 or 25 μm orifice ruby straight hole type nozzle 324, Bird Precision, Waltham, MA). The moisture trap 212 is placed between cartridge 100 and stage 1 gas regulator 210. The motor pump 220 is used as a vacuum pump to remove gas from the manifold to exhaust system or to NO scrubber before initiation and stopping the device. Unless otherwise indicated, parts not specifically referenced in relation to FIG. 14 are the same as in FIG. 13.

Stage 1 gas regulator 210 restricts the NO-cartridge outlet pressure to a maximum of 200 kPa. Stage 2 regulator 218 electronically controls the differential gauge pressure level from 0.25 kPa to 200 kPa in the NO-manifold 214. NO pressure in the cartridge 100 is in the range of 500 kPa to 1500 kpa. Using both stage 1 and 2 pressure regulators can reduce the cartridge NO pressure to a minimum level of 0.25 kPa at the delivery point of the nozzle 324 orifice. The isolation valve 216 electronically controls the NO exit from NO-manifold 214 to the mixing chamber 300, where the gas is delivered via nozzle assembly 320. The isolation valve also prevents the entry of carrier gas into the NO manifold 214. If the carrier gas pressure is higher than manifold pressure, the program automatically closes the isolation value 216. As an alternative to oxygen, the system 3000 can optionally use air as a carrier gas by integrating a small air pump to supply the source of air (not shown). An injector 376 that is combined with a gas flow sensor 260 (not shown) can be placed at the gas outlet of a ventilator inspiratory tube 370 or high flow nasal cannula (HFNC) to deliver the therapeutic mixed gas to the patient. The injector nozzle 376 orifice size is from 100 μm to 1000 μm to mix NO rapidly with inspired oxygen to minimize the NO₂ formation. The flow sensor 260 determines the carrier gas flow rate in milliseconds and communicate to CPU 500. The concentrated NO is diluted in nitrogen stream or air (0.5 to 1 L/min) to a lower concentration (<1000 ppm) in the mixing chamber 300 prior to introducing it into inspiratory tube 370 through the injector 376.

Operation of system 3000: The pressurized NO-cartridge 100 is inserted into a cartridge receptacle in the system. The user sets the desired dose on a user interface connected to CPU 500. The flow sensor 260 measures the flow rate of inspired O₂ and communicates to the CPU 500. The calculation for dosing of NO is as follows (see FIGS. 12A and 12B for details): Set dose×established pressure difference/at known NO concentration×carrier gas flow rate+carrier gas pressure. The stock NO concentration in the cartridge 100 is always constant. NO that is generated in the reaction vessel has a concentration of about 90 to 98%. Carrier gas flow rate is converted to pressure by CPU 500. The CPU 500 electronically communicates the calculated values to the pressure regulator to regulate the desired pressure on the NO-manifold 214 to dispense NO. The stage 2 pressure regulator 218 adjusts the pressure difference within seconds to the inspired O₂ flow rates. Therefore, the NO doses will be constant irrespective of flow rate changes in the ventilator and HFNC.

Advantageously, the cartridge 100 without chemicals in it can be used to fill the medical grade NO to the concentration of 2 to 100% at the pressure of 50 to 500 psi. These pre-filled cartridges can be used in the device as a source of inhaled NO. The system can use cartridges 100 that are pre-filled with medical grade NO in the range of 2 to 100% concentration at the pressure of 50 to 500 psi. The efficiency of NO dosing is more efficient with diluted stock NO because the pressure difference between NO and carrier gas is high as a result it is easy to regulate the NO dosing with pressure regulator. In addition, the two stages dilution (e.g., in the mixing chamber and again when mixed with inspired O₂) is not required for using diluted NO of 2 to 20% because the NO₂ formation would be within the safety limits. The injector module 376 in the inspiratory tube can be replaced with a mixing chamber 300 that is integrated with a gas flow sensor.

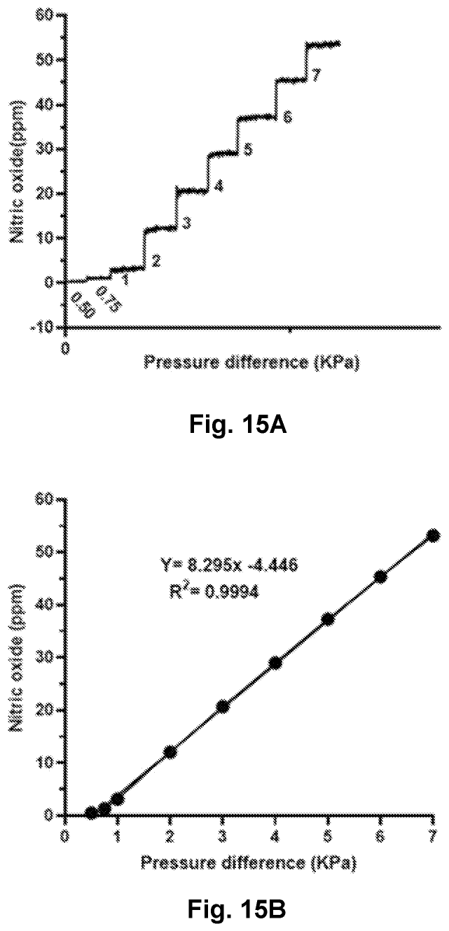

To test NO dosing, a prototype was built based on the designs shown In FIGS. 13 and 14. The device dashboard (e.g., a touch screen) has electronically controllable buttons for start/stop, carrier gas flow rate settings, manifold (NO gas) and carrier gas pressure indicators, and a manifold pressure regulator for NO dosing. The NO and NO₂ levels are measured in the combined NO and oxygen gas by an external Sievers NO analyzer and CAPS NO₂ analyzer, respectively. The carrier gas flow is set at the desired level. Upon starting the simulation, the device undergoes a self-check and initiates the vacuum pump to remove the gas present in the gas lines. The carrier gas flow is initiated, and the pressure level is displayed on a touchscreen. The initial NO gas pressure is set equal to the carrier gas flow pressure. After an indication of “waiting for NO gas” is shown, the pressurized NO cartridge is connected to the gas inlet socket. Once the NO reaches the set pressure on the manifold, the mixing chamber valve opens for delivery of NO through a nozzle. The NO delivery is zero when the pressure difference between NO and carrier gas is zero. Then the NO pressure is increased by using the touchscreen pressure regulator control buttons starting from 0.25 to 7 kPa (relative gauge pressure to carrier gas). The NO signal response to the pressure change is immediate (<2 seconds) and quickly stabilizes to the peak level (FIG. 15A). The NO is injected into the carrier gases by a 10-μm nozzle. NO is injected into the carrier gas when its pressure is above that of the carrier gas. This increase in pressure difference is linearly correlated (R²=0.9993) with increasing NO dose (FIG. 15B). The linear regression analysis of this plot shows that a pressure difference of 1 kPa is equivalent to the dose of 8.3 ppm NO (FIG. 15B). The sensitivity of the pressure regulator is 0.25 kPa, which reflects an NO dosing sensitivity of 2.06 ppm. In the future, the sensitivity can be increased using a more sensitive pressure regulator that can increase the sensitivity for dosing of NO to 0.5 ppm and compatible to 100% NO gas. The off-the-shelf Clippard pressure regulator used in the prototype works well for testing the concept of metering NO based on the pressure difference between NO and carrier gas.

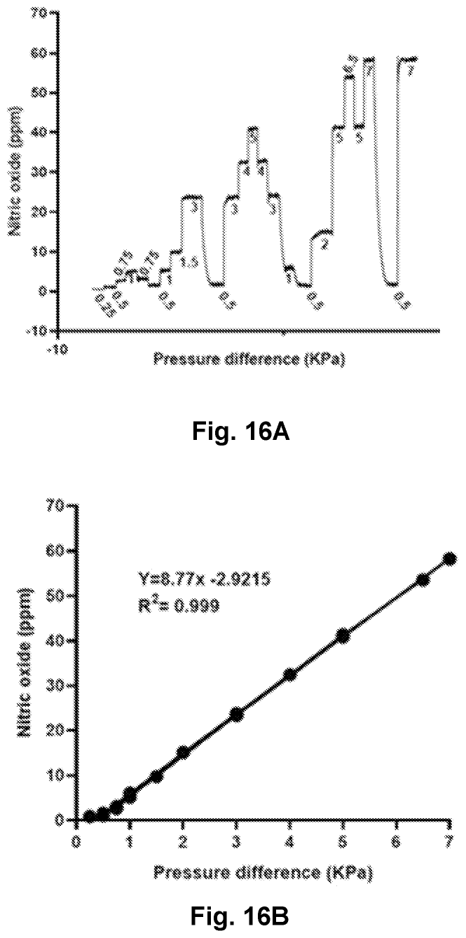

FIGS. 16A and 16B show the dose modulation during the therapy. The NO dose may need to be adjusted at any point during patient therapy. The dose can be altered by changing the manifold NO pressure with pressure regulator controls. Therefore, we investigated the response of NO dose to the pressure change during delivery at the fixed carrier gas flow rate of 4 L/min. As shown in FIG. 16, changing the NO pressure in either a positive or negative direction affected NO dose levels. The response occurred within a couple of seconds and stabilized quickly at the increased or decreased peak level. The response of NO dosing correlated (R=0.999) well with the pressure difference. These results suggest that NO dose can be changed any time to the desired level by regulating the pressure difference.

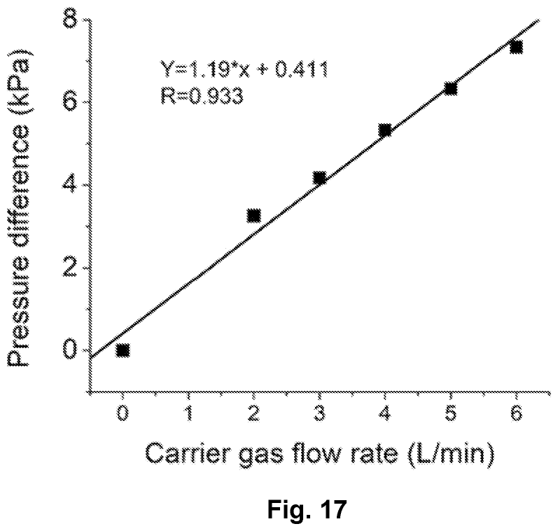

FIG. 17 further demonstrates that pressure difference is correlated to carrier gas flow rate at the fixed NO dose. Inspired oxygen flow rates are increased or decreased during iNO therapy based on patient requirements. These changes immediately affect NO dose levels because NO dilution level changes. However, NO dosing needs to be constant irrespective of inspired oxygen flow rate. Dose changes can be corrected to the set level by regulating the pressure difference. FIG. 17 shows the pressure difference corrections used to compensate for the change of carrier gas flow rates to maintain a fixed 20 ppm NO dose. The NO dosing is initiated at 20 ppm at the carrier gas flow rate of 4 L/min. The purpose of the experiment was to maintain this NO dose while changing the carrier flow rates. The flow rate was increased or decreased in 0.5 L intervals and was reflected immediately in the NO dose level. This change in dose was adjusted back to 20 ppm by increasing or decreasing the pressure difference as needed. The plot of flow rate against NO dose is linearly correlated (R=0.933). The linear regression analysis of this plot shows that 1 L/min carrier gas flow rate is equal to the pressure difference 1.6 kPa at the fixed internal diameter and length of gas lines. These data can be used in software that will allow the system to adjust the dosing electronically within seconds to prevent changes in NO dose.

In our previous work, NO₂ levels in the combined NO and O₂ gas were measured upon exiting the mixing chamber from a 25-μm nozzle. The present system uses a 10-μm NO nozzle 324. NO₂ and NO levels are measured simultaneously in the mixed gas that exits the mixing chamber. NO₂ levels in the nitrogen carrier gas indicate the NO₂ present in the stock NO and/or formed while transferring NO from the cartridge to the mixing chamber. Mixing NO gas with air and 100% O₂ results in formation of NO₂ with increasing NO concentration. NO₂ formation is within the safety limits of <1 ppm even at the maximum permitted dose of 80 ppm NO. The rate of NO reaction with O₂ to generate NO₂ is the square of the NO concentration and proportional to the O₂ concentration. As seen in the Table 3, increasing NO concentration from 20 ppm to 40 ppm and 80 ppm generates only 3-fold and 9-fold NO₂, respectively, rather than 4-fold and 16-fold. The possible explanation for this result is that NO is delivered into the mixing chamber at the fixed orifice of the nozzle. As the NO injecting volume increases, the flow velocity of NO gas that is emerging from the nozzle's orifice also increases, thereby resulting in more rapid mixing with O₂ (or other carrier gas). The more rapid mixing in turn decreases the dilution time, thereby decreasing NO reaction with O₂ to generate NO₂. Therefore, NO₂ formation does not pose a problem even at higher doses of NO in pressure-mediated NO delivery because it does not increase exponentially with increasing the NO concentration. Table 3 shows the NO₂ levels in the NO and carrier gas mixture.

| NO (ppm) | NO₂ (ppb) | | | Subtracted from | |
|---|---|---|---|---|---|
| Carrier | Nitrogen | Air | Oxygen | baseline NO₂ (ppb) | |
| gas | (% O₂) | (21% O₂) | (100%) | Air | Oxygen |
| 5 | 5 ± 1 | 11 ± 2.4 | 24 ± 3.8 | 6 ± 1.2 | 19 ± 3.2 |
| 10 | 10.7 ± 2.3 | 21 ± 5 | 41 ± 7.8 | 10.3 ± 3.3 | 30.3 ± 6.4 |
| 20 | 17.2 ± 5 | 38 ± 7.8 | 64 ± 12.6 | 20.8 ± 5.2 | 56.8± |
| 40 | 28.2 ± 6.4 | 84 ± 13.2 | 168 ± 22 | 55.8 ± 12.5 | 139.8 ± 21 |
| 60 | 42.6 ± 8.8 | 121 ± 18 | 324 ± 34.8 | 78.4 ± 18 | 281.4 ± 29.5 |
| 80 | 54.2 ± 12.5 | 172 ± 25 | 480 ± 50.4 | 118 ± 16.7 | 425.8 ± 43 |

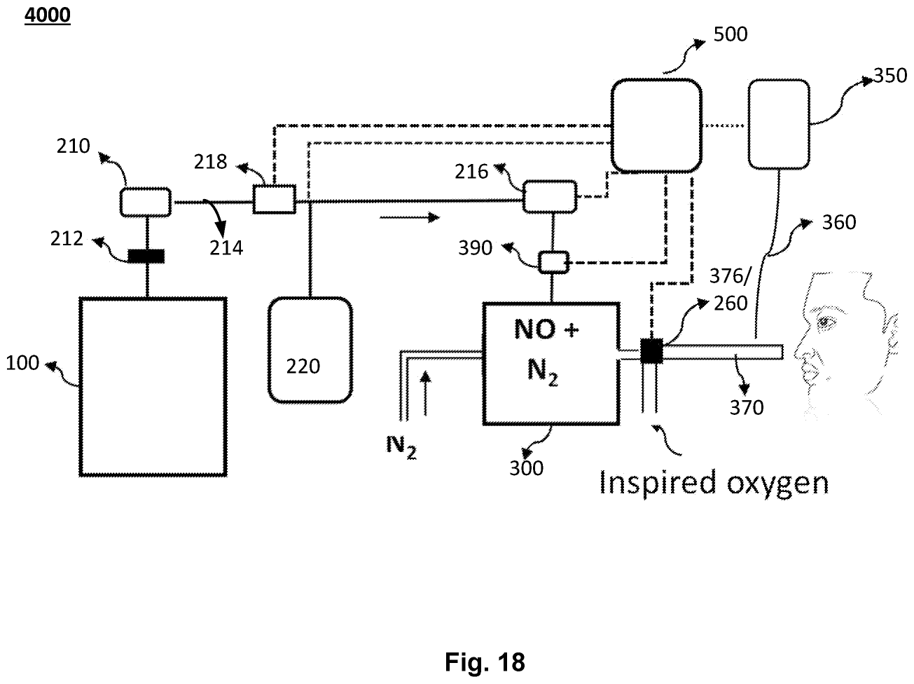

Proportional valve NO delivery: FIG. 18 is a schematic illustration of another embodiment of an iNO system 4000 using a proportional valve for the dosing of NO. The proportional valve automates the flow of gas in proportion to the input current. Hence, it controls the flow of gas to the desired level by timing on/off cycles. The proportional valve is used in existing cylinder-based iNO therapy to dilute 800 ppm NO. However, in the present system, 90 to 98% % NO (0.9 to 0.98 million ppm) needs to be diluted. In other words, our system needs to inject microliter volumes of NO compared to milliliter volumes in the existing system. The proportional valves used in existing systems are not capable of dosing small volumes of high-concentration NO and simply scaling down the proportional valve would not address the precise pressure needed to deliver the NO into the carrier gas at a rate sufficient to prevent formation of toxic NO₂. Here, commercially available ultra-miniature proportional valves (Lee Company, CT, USA) that can dispense precise volumes in the microliter range are combined with small delivery nozzles (e.g., 10 or 25 micron) to solve this problem. System 4000, shown in FIG. 18, is similar to the pressure mediated NO delivery device in FIG. 14, except a proportional valve is introduced for NO dosing instead of the pressure regulator. Parts not specifically referred to in the text related to FIG. 18 correspond to the same parts in FIG. 14. Because use of the proportional valve requires that the manifold NO pressure be constant and higher than the mixing chamber carrier gas pressure, the software program (in CPU 500) that controls pressure is modified. Turning to FIG. 18, the NO cartridge 100 is in fluid communication with the carrier gas in the mixing chamber 300 via stage 1 pressure regulator 210, stage 2 pressure regulator 218, isolation valves 216 and proportional valve 390. Stage 1 gas regulator 210 restricts the NO-cartridge outlet pressure to a maximum of 30 psi. The motor pump 220 is used as a vacuum pump to remove gas from the manifold to exhaust system or NO scrubber. Stage 2 regulator 218 electronically controls the differential gauge pressure level from 1 psi to 30 psi. NO manifold 214 pressure is set at the lowest possible carrier gas NO value that maintains a positive differential pressure between manifold 214 and mixing chamber 300 (across all anticipated carrier gas pressures) by the stage 2 regulator 218. This NO pressure is maintained at a constant in the manifold irrespective of NO delivery. All doses of NO are delivered at this fixed pressure difference by modulating the proportional valve 390 pulse width time via flow sensor 260 and CPU 500. NO is diluted in two stages. First, NO is diluted to 200 to 1000 ppm based on the dosing requirements in a fixed volume of N₂ (0.5 L to 1 L/min) in the mixing chamber 300. Second, this diluted NO is introduced into inspiratory tubing 370 through the injector 376 combined with gas flow sensor 260. The injector nozzle 376 orifice size is 100 μm to 1000 μm to mix NO rapidly with inspired oxygen to minimize the NO₂ formation. Parts 218, 216, 390, 220, and 260 are connected electronically to the CPU 500. The software system: The flow sensor 260 measures O₂ flow rate and communicates to the CPU 500. The CPU 500 calculates the dose based on the user input dose and commands the proportional valve 390 to inject NO into the mixing chamber 300. The stock NO concentration delivered from cartridge 100 remains constant. The calculation is as follows: Set NO dose (ppm)/stock NO (ppm)×carrier gas flow rate (L/min). NO is diluted in a stream of nitrogen inside mixing chamber 300 at the fixed flow rate to a lower concentration <1000 ppm before it is introduced into inspired gas. This device can be used in hospitals for ventilators and high flow nasal cannula continuous positive airway pressure iNO therapies. In traditional iNO therapy, a significant amount of NO gas is consumed because the inspired gas flow rate is usually as high as 60 L/min.

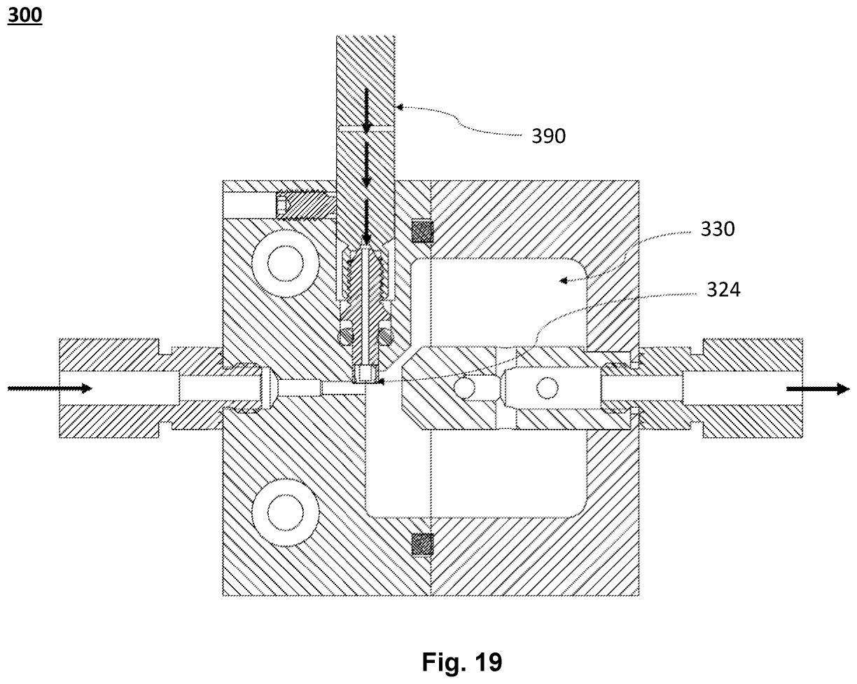

FIG. 19 shows a mixing chamber 300 equipped with an ultra-miniature proportional valve, as described in FIG. 18. The mixing chamber 300 used for pressure-mediated NO dosing has been modified for proportional valve-mediated NO dosing. The small size and cylindrical form-factor of the proportional valve allow it to be nearly a direct replacement to the current orifice nozzle. The first iteration of this proportional valve design uses the industry standard model of the valve; therefore, a custom stainless-steel nozzle attaches to the end of the valve to create the 10-μm, 25-μm, or 50-μm orifice. The nozzle and valve assembly are inserted into the mixing chamber so that the orifice is placed in the stream of the carrier gas; this end geometry is nearly identical to that of the pressure measure-mediated NO delivery. The fundamental difference from pressure-mediated delivery is that NO dosing is controlled by changing the pulse width (PW) frequency of the valve at a fixed NO pressure. The user interface has an additional option for increasing or decreasing the PW frequency of the proportional valve to meter the NO gas. A custom valve assembly can be substituted to minimize the amount of dead volume between the internal seal of the valve and orifice, allowing the system to dynamically respond to changes in the carrier gas flow rate more quickly than using a standard commercial proportional valve.

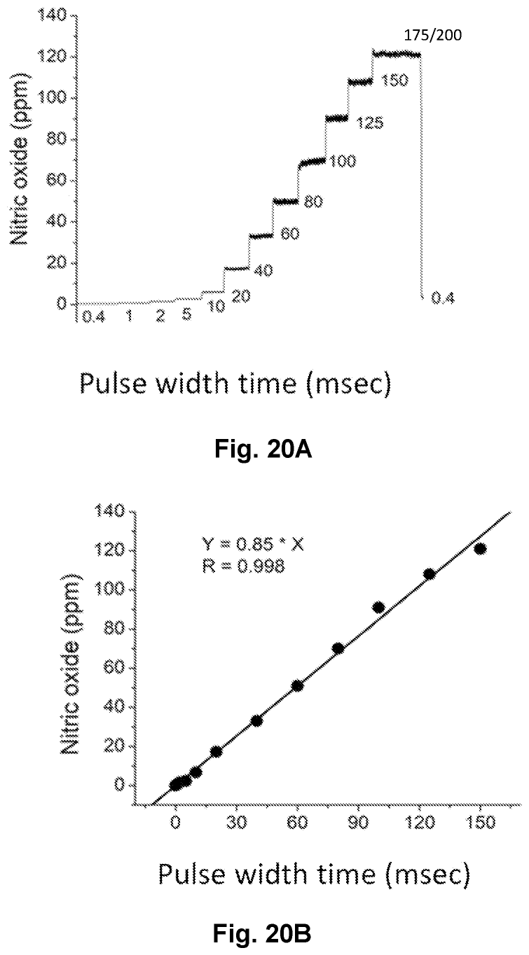

NO delivery increases with increasing the pulse width (valve held open longer per cycle) rate of the valve. In a particular embodiment, the carrier gas flow rate is set at 5 L/min and initiates the procedure. The mixing chamber gas pressure that indicates the carrier gas pressure is 17 psi at this flow rate. The default PW is always 0.4 milliseconds/cycle. It allows NO delivery when its threshold pressure limit is crossed. The manifold pressure (NO pressure) is increased by using the pressure regulator controls on the touchscreen until the NO signal is seen on the NO analyzer program. At these settings, the manifold threshold pressure for NO is 22 psi. The presumption is that all the doses of NO can be delivered at this fixed manifold pressure. The pressure difference between the carrier gas and NO gas is 5 psi, at which the NO dosing is <0.1 ppm. In this experiment, we increased PW frequency in a stepwise manner while continuously recording NO signals. As shown in FIGS. 20A and 20B, NO dosing increases with increasing PW rate. The NO signal response to changes in the PW is very quick and stabilizes within a couple of seconds at the peak level. This delivery of NO is stable as long as the manifold pressure is maintained constant by a pressure regulator. The increase in PW rate is linearly correlated (R=0.998) with increasing NO dose. A 1 millisecond PW rate is equal to delivery of 0.85 ppm NO based on the linear regression plot (FIGS. 20A and 20B).

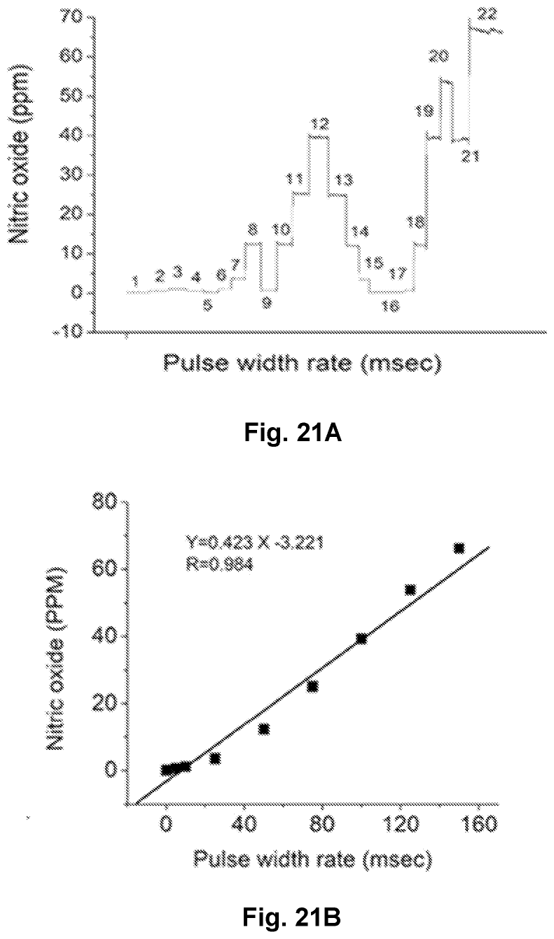

FIGS. 21A and 21B shows NO dose modulation during therapy using the proportional valve system. NO dosing frequently must be altered during therapy according to patient requirements. It can be changed/adjusted by modulating the PW frequency rate. Therefore, we investigated how the NO dose responds to a PW rate change at the fixed carrier gas flow rate. As shown in FIGS. 21A and 21B, a positive or negative change in PW was precisely reflected in NO dose levels within a couple of seconds and stabilized at the peak dose level. Dose adjustments back and forth did not affect the delivery of NO per unit change of PW, as shown by the linear fit (0.984) of all the points of pulse rate vs NO dose.

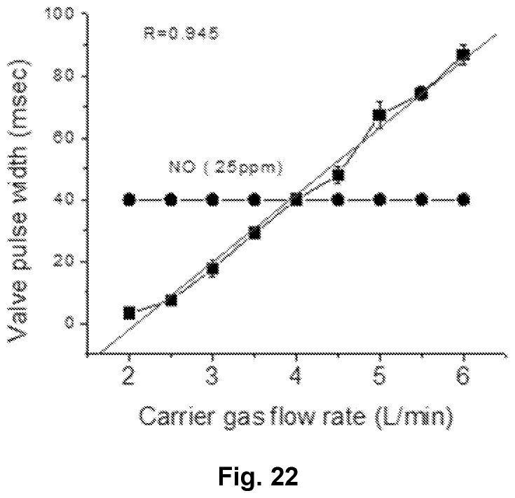

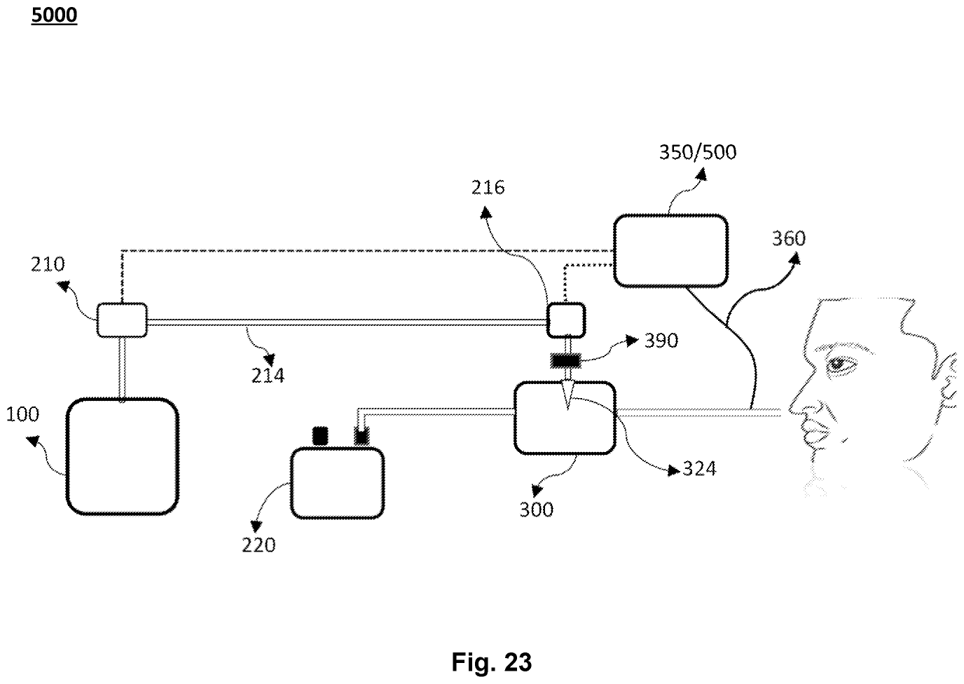

FIG. 22 shows the carrier gas flow rate is proportional to pulse width rate. Any change in carrier gas flow rate during therapy is reflected in the NO dose levels. However, NO must be maintained at a constant dose throughout therapy, regardless of carrier gas flow rates. These changes in the NO dose level can be corrected by adjusting the PW frequency that corresponds to the change in flow rate. FIG. 22 shows the results of NO dose corrections for the set level to the altered carrier gas flow rates. NO dose was set at 25 ppm at the initial carrier gas flow rate of 4 L/min. The required PW rate for this dose is 40 milliseconds. In this experiment, the carrier gas flow rate was increased from 4 L/min to 6 L/min in 0.5 L/min intervals. These changes in carrier gas flow rate decrease NO dose level because NO dilution increases. However, the decrease in NO dose was corrected to the set level by increasing the PW from 40 milliseconds to 86 milliseconds. Increasing the PW rate (valve held open longer per cycle) results in injection of more NO gas. Similarly, decreasing the carrier gas flow rate will increase the NO dose as a result of less dilution. Again, the change was corrected by decreasing the PW rate (valve held open for less time per cycle). Our proportional valve combined with a 10-μm or 25-μm nozzle regulates the NO dosing precisely by injecting NO in microliter volumes. At the prototype stage, all dosing functions were adjusted manually, however, software in the CPU can be used to control the NO dosing electronically through the user interface.

Portable iNO therapy system: FIG. 23 is a schematic of one example of a portable system 5000. It is a hybrid model of the NO delivery systems described above. The system 5000 has an option to use a proportional valve or pressure mediated NO dosing. This device is meant for use in and out of hospitals (homes, patients transporting vehicles, and ambulatory patients) for NO delivery via nasal/facemask interface. The hospital-based device that is integrated with a proportional valve with nozzle system (FIG. 18) is simplified to a smaller, lightweight version for portable use. The NO-cartridge 100 size can be reduced to <50 L volume NO, which is sufficient for 24 hours of therapy. NO-generating chemicals are stored in the original cartridge 100 to generate NO gas on-demand. Alternatively, the same cartridge can also be used to fill with medical grade NO (5% to 100% concentration) to use as a source of iNO. In this case, pre-filled pressurized NO cartridges are shipped to hospitals. The NO in the cartridge 100 is in fluid communication with inspired air in the mixing chamber 300 via pressure regulator 210, NO gas line 214, isolation valve 216, proportional valve 390 and nozzle 324. The proportional valve 390 that is integrated with nozzle 324 in the mixing chamber 300 can be operated using either the proportional valve combined with nozzle or only nozzle-based NO dosing. When the proportional valve mode is turned off, the proportional valve is set to a completely open position; as result the NO is delivered via nozzle controlled by pressure regulator 210. The pressure regulator 210 either maintains the constant NO pressure for proportional valve mediated delivery or maintains the desired pressure difference in the manifold for nozzle-mediated delivery. Pressure regulator 210, isolation valve 216 and proportional valve 390 have communication with CPU 500. NO and NO₂ levels are measured constantly in sampling gas in gas sampling line 360 with the gas sensor system 350. The gas sensor system 350 is a safety system integrated with CPU 500 to alert with an alarm or shut-off of delivery completely if NO or NO₂ exceed the safety limit. The system is integrated with an air pump 220 to provide a source of atmospheric air for a carrier gas (<10 L/min) with an option for cylinder source O₂. The recommended dose of NO is 20 ppm or less to treat pulmonary hypertension. Therefore, the doses can be fixed at such as 5 ppm, 10 ppm, or 20 ppm at the fixed rate of carrier gas flow rates. Fixed dosing can simplify the software development and safety system used for these NO doses and carrier gas flow rates.

### Aspects of the Disclosure

The present disclosure will be better understood upon reading the following numbered aspects, which should not be confused with the claims. Any of the numbered aspects below can, in some instances, be combined with aspects described elsewhere in this disclosure and such combinations are intended to form part of the disclosure.

Aspect 1. A device for on-demand generation of NO, the device comprising:
a reaction vessel and a liquid storage component, wherein the reaction vessel comprises a liquid injection port and a gas exit port;
wherein the liquid injection port and a gas exit port are each fitted with a self-sealing plug connectors pressure-tolerant up to 500 PSI such that the reaction vessel is self-sealing and pressure-tolerant up to 500 PSI; and
wherein the liquid storage component is removably coupled to the reaction vessel at the liquid injection port via a socket connector.

Aspect 2. The device according to aspect 1, wherein the liquid storage component is a gas-tight, pressure resistant syringe.

Aspect 3. The device according to aspect 1, wherein the liquid storage component is a gas-tight, pressure resistant vessel.

Aspect 4. The device according to claim any of the preceding aspects, wherein the reaction vessel is a stainless-steel vessel.

Aspect 5. The device according to any of the preceding aspects, wherein the reaction vessel further comprises one or more reactants in solid or powder form.

Aspect 6. The device according to aspect 5, wherein upon injection of a liquid from the liquid storage component into the reaction vessel to initiate formation of NO gas, the liquid storage component is decoupled from the reaction vessel such that the NO gas is sealed in the reaction vessel and the reaction vessel is pressurized by the NO gas.

Aspect 7. A mixing device (also referred to as a chamber) for mixing NO gas with a carrier gas, the device comprising:
a carrier gas inlet, an NO inlet, a mixing space, and a gas outlet,
wherein the carrier gas inlet and the NO inlet are positioned such that NO gas injected into the mixing chamber from the NO inlet is injected into a carrier gas streamed from the carrier gas inlet to form a mixed gas, and wherein the mixed gas exits the mixing device through the gas outlet.

Aspect 8. The mixing device according to aspect 7, wherein the NO inlet is fitted with a nozzle having an inner diameter of 10 μm to 50 μm.

Aspect 9. The mixing device according to aspect 7, wherein the NO inlet is fitted with a nozzle having an inner diameter of 10 μm.

Aspect 10. The mixing device according to aspects 7-9, further comprising an obstruction in the mixing space, wherein the obstruction diverts the mixed gas around the chamber to facilitate further mixing before the mixed gas exits through the gas outlet.

Aspect 11. The mixing device according to aspects 7-10, wherein the carrier gas inlet channel cross-sectional area is reduced by 40% into a slot channel that is perpendicular to the nozzle orifice surface to increase the flow velocity of carrier gas.

Aspect 12. A two-stage dilution system for delivering NO to a patient, the system comprising:
a removable NO gas cartridge in fluid communication with a gas mixing chamber and a CPU,
wherein the gas mixing chamber comprises a carrier gas inlet, an NO inlet, a mixing space, and a gas outlet;
wherein the NO inlet comprises an NO nozzle having an internal diameter of about 10 μm to 50 μm,
wherein concentrated NO gas is delivered from the NO gas cartridge to the gas mixing chamber via a gas manifold, and wherein the pressure of the NO gas in the manifold is controlled by an electronic pressure regulator in communication with the CPU,
wherein a carrier gas selected from air or N₂ flows into the carrier gas inlet at a fixed flow rate,
and wherein the NO nozzle injects the NO into a stream of carrier gas to mix the gas in the mixing chamber to form a first diluted mixed gas comprised of NO diluted in the carrier gas, wherein the mixed gas comprises less than 1000 ppm of NO and less than 25 ppb of NO₂.

Aspect 13. The system according to aspect 12, further comprising an oxygen source coupled the gas outlet via a flow sensor, wherein the flow sensor measures the flow rate of inspired O₂ and communicates to the CPU to inject the desired NO dose of the first diluted mixed gas into the O₂ for delivery of a second dilute gas to a patient via an inspiratory tube at a desired NO dose, and wherein the desired NO dose is about 1 ppm to 80 ppm.

Aspect 14. The system according to aspect 12 or 13, wherein the pressure regulator regulates the NO gas flow based on instructions from the CPU, where the NO gas flow rate is based on a pressure difference between the carrier gas and the NO gas to result in a desired dilution of the concentrated NO gas.

Aspect 14b. The system according to any of aspects 12-14, further comprising a proportional valve controlling a flow of NO entering the mixing chamber, wherein the pressure regulator maintains a constant pressure of NO gas in the manifold, and wherein the CPU calculates a pulse width time of the proportional valve based on the desired NO dose and carrier gas flow rate.

Aspect 15. The system according to any of aspects 12-14, wherein the concentrated NO gas in the gas cartridge has a concentration of about 10% to 100%.

Aspect 16. The system according to any of aspects 12-15, further comprising an exhaust system, the exhaust system comprising a vacuum pump connected to the manifold.

Aspect 17. The system according to any of aspects 12-16, further comprising a gas sensor system connected to the inspiratory tube, wherein the gas sensor system measures NO, NO₂ and % O₂ content in the inspired gas.

Aspect 18. The system according to aspect 17, wherein the gas sensor system is in communication with the CPU and provides an alert or stops the system if the NO and NO₂ content exceeds a predetermined threshold.

Aspect 19. The system according to any of aspects 12-18, wherein the pressure regulator regulates the pressure of the NO in the manifold between about 0.25 kPa and 200 kPA.

Aspect 20. The system according to any of aspects 12-19, wherein the NO pressure in the NO gas cartridge is about 50 psi to about 220 psi.
a removable NO gas cartridge in fluid communication with a gas mixing chamber and a CPU,
wherein the gas mixing chamber comprises a carrier gas inlet, an NO inlet, a mixing space, and a gas outlet;

Aspect 21. A system for delivering NO to a patient, the system comprising:
wherein the NO inlet comprises an NO nozzle having an internal diameter of about 10 μm to 50 μm
wherein concentrated NO gas is delivered from the NO gas cartridge to the gas mixing chamber via a gas manifold, and wherein the pressure of the NO gas in the manifold is controlled by an electronic pressure regulator in communication with the CPU,
and wherein the NO nozzle injects the NO into a stream of carrier gas to mix the gas in the mixing chamber to form a mixed gas comprised of NO diluted in carrier gas, wherein the mixed gas comprises from 1 ppm to 1000 ppm)

Aspect 22. The system according to aspect 21, wherein the pressure regulator regulates the NO gas flow based on instructions from the CPU, where the NO gas flow rate is based on a pressure difference between the O₂ and the NO gas to result in a desired dilution of the concentrated NO gas.

Aspect 23. The system according to aspects 21-22, further comprising a proportional valve controlling a flow of NO entering the mixing chamber, wherein the pressure regulator maintains a constant pressure of NO gas in the manifold, and wherein the CPU calculates a pulse width time of the proportional valve based on the desired NO dose and O₂ flow rate.

Aspect 24. The system according to any of aspects 21-23, wherein the concentrated NO gas in the gas cartridge has a concentration of about 10% to 100%.

Aspect 25. The system according to any of aspects 21-24, further comprising an exhaust system, the exhaust system comprising a vacuum pump connected to the manifold.

Aspect 26. The system according to any of aspects 21-25, further comprising a gas sensor system connected to the gas exit, wherein the gas sensor system measures NO, NO₂, and O₂ content in the inspired gas.

Aspect 27. The system according to any of aspects 21-26, wherein the gas sensor system is in communication with the CPU and provides an alert or stops the system if the NO and NO₂ content exceeds a predetermined threshold.

Aspect 28. The system according to any of aspects 21-27, wherein the pressure regulator regulates the pressure of the NO in the manifold between about 0.25 kPa and 200 kPA.

Aspect 29. The system according to any of aspects 21-2, wherein the NO pressure in the NO gas cartridge is about 50 psi to about 220 psi.

Aspect 30. The system according to any of aspects 21-29, wherein the system is a portable system.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. In an embodiment, “about 0” can refer to 0, 0.001, 0.01, or 0.1. In an embodiment, the term “about” can include traditional rounding according to significant figures of the numerical value. In addition, the phrase “about ‘x’ to ‘y’” includes “about ‘x’ to about ‘y’”.

Disjunctive language such as the phrase “at least one of X, Y, or Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

As used herein, the terms “about,” “approximately,” “at or about,” and “substantially equal” can mean that the amount or value in question can be the exact value or a value that provides equivalent results or effects as recited in the claims or taught herein. That is, it is understood that amounts, sizes, measurements, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In general, an amount, size, measurement, parameter or other quantity or characteristic is “about,” “approximate,” “at or about,” or “substantially equal” whether or not expressly stated to be such. It is understood that where “about,” “approximately,” “at or about,” or “substantially equal” is used before a quantitative value, the parameter also includes the specific quantitative value itself, unless specifically stated otherwise.

Where a range is expressed, a further aspect includes from the one particular value and/or to the other particular value. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

It should be emphasized that the above-described embodiments of the present disclosure are merely possible examples of implementations and are set forth only for a clear understanding of the principles of the disclosure. Many variations and modifications may be made to the above-described embodiments of the disclosure without departing substantially from the spirit and principles of the disclosure. All such modifications and variations are intended to be included herein within the scope of this disclosure.

Although embodiments have been described herein in detail, the descriptions are by way of example. The features of the embodiments described herein are representative and, in alternative embodiments, certain features and elements may be added or omitted. Additionally, modifications to aspects of the embodiments described herein may be made by those skilled in the art without departing from the spirit and scope of the present invention defined in the following claims, the scope of which are to be accorded the broadest interpretation so as to encompass modifications and equivalent structures.

1-31. (canceled)

32. A two-stage dilution system for delivering NO to a patient, the system comprising:
a removable NO gas cartridge in fluid communication with a gas mixing chamber and a CPU,
wherein the gas mixing chamber comprises a carrier gas inlet, an NO inlet, a mixing space, and a gas outlet;
wherein the NO inlet comprises an NO nozzle having an internal diameter of about 10 μm to 50 μm,
wherein concentrated NO gas is delivered from the NO gas cartridge to the gas mixing chamber via a gas manifold, and wherein the pressure of the NO gas in the manifold is controlled by an electronic pressure regulator in communication with the CPU,
wherein a carrier gas selected from air or N₂ flows into the carrier gas inlet at a fixed flow rate,
and wherein the NO nozzle injects the NO into a stream of carrier gas to mix the gas in the mixing chamber to form a first diluted mixed gas comprised of NO diluted in the carrier gas, wherein the mixed gas comprises less than 1000 ppm of NO and less than 25 ppb of NO₂.

33. The system according to claim 32, further comprising an oxygen source coupled the gas outlet via a flow sensor, wherein the flow sensor measures the flow rate of inspired O₂ and communicates to the CPU to inject the desired NO dose of the first diluted mixed gas into the O₂ for delivery of a second dilute gas to a patient via an inspiratory tube at a desired NO dose, and wherein the desired NO dose is about 1 ppm to 80 ppm.

34. The system according to claim 32, wherein the pressure regulator regulates the NO gas flow based on instructions from the CPU, where the NO gas flow rate is based on a pressure difference between the carrier gas and the NO gas to result in a desired dilution of the concentrated NO gas.

35. The system according to claim 32, further comprising a proportional valve controlling a flow of NO entering the mixing chamber, wherein the pressure regulator maintains a constant pressure of NO gas in the manifold, and wherein the CPU calculates a pulse width time of the proportional valve based on the desired NO dose and carrier gas flow rate.

36. The system according to claim 32, wherein the concentrated NO gas in the gas cartridge has a concentration of about 10% to 100%.

37. The system according to claim 32, further comprising an exhaust system, the exhaust system comprising a vacuum pump connected to the manifold.

38. The system according to claim 32, further comprising a gas sensor system connected to the inspiratory tube, wherein the gas sensor system measures NO, NO₂ and % O₂ content in the inspired gas.

39. The system according to claim 38, wherein the gas sensor system is in communication with the CPU and provides an alert or stops the system if the NO and NO₂ content exceeds a predetermined threshold.

40. The system according to claim 32, wherein the pressure regulator regulates the pressure of the NO in the manifold between about 0.25 kPa and 200 kPA.

41. The system according to claim 32, wherein the NO pressure in the NO gas cartridge is about 50 psi to about 220 psi.

42. A system for delivering NO to a patient, the system comprising:
a removable NO gas cartridge in fluid communication with a gas mixing chamber and a CPU,
wherein the gas mixing chamber comprises a carrier gas inlet, an NO inlet, a mixing space, and a gas outlet;
wherein the NO inlet comprises an NO nozzle having an internal diameter of about 10 μm to 50 μm
wherein concentrated NO gas is delivered from the NO gas cartridge to the gas mixing chamber via a gas manifold, and wherein the pressure of the NO gas in the manifold is controlled by an electronic pressure regulator in communication with the CPU,
and wherein the NO nozzle injects the NO into a stream of carrier gas to mix the gas in the mixing chamber to form a mixed gas comprised of NO diluted in carrier gas, wherein the mixed gas comprises from 1 ppm to 1000 ppm.

43. The system according to claim 42, wherein the pressure regulator regulates the NO gas flow based on instructions from the CPU, where the NO gas flow rate is based on a pressure difference between the O₂ and the NO gas to result in a desired dilution of the concentrated NO gas.

44. The system according to claim 42, further comprising a proportional valve controlling a flow of NO entering the mixing chamber, wherein the pressure regulator maintains a constant pressure of NO gas in the manifold, and wherein the CPU calculates a pulse width time of the proportional valve based on the desired NO dose and O₂ flow rate.

45. The system according to claim 42, wherein the concentrated NO gas in the gas cartridge has a concentration of about 10% to 100%.

46. The system according to claim 42, further comprising an exhaust system, the exhaust system comprising a vacuum pump connected to the manifold.

47. The system according to claim 42, further comprising a gas sensor system connected to the gas exit, wherein the gas sensor system measures NO, NO₂, and O₂ content in the inspired gas.

48. The system according to claim 42, wherein the gas sensor system is in communication with the CPU and provides an alert or stops the system if the NO and NO₂ content exceeds a predetermined threshold.

49. The system according to claim 42, wherein the pressure regulator regulates the pressure of the NO in the manifold between about 0.25 kPa and 200 kPA.

50. A mixing device for mixing NO gas with a carrier gas, the device comprising:
a carrier gas inlet, an NO inlet, a mixing space, and a gas outlet,
wherein the carrier gas inlet and the NO inlet are positioned such that NO gas injected into the mixing chamber from the NO inlet is injected into a carrier gas streamed from the carrier gas inlet to form a mixed gas, and wherein the mixed gas exits the mixing device through the gas outlet.

51. A device for on-demand generation of NO, the device comprising:
a reaction vessel and a liquid storage component, wherein the reaction vessel comprises a liquid injection port and a gas exit port;
wherein the liquid injection port and a gas exit port are each fitted with a self-sealing plug connectors pressure-tolerant up to 500 PSI such that the reaction vessel is self-sealing and pressure-tolerant up to 500 PSI; and
wherein the liquid storage component is removably coupled to the reaction vessel at the liquid injection port via a socket connector.
